(19) United States
(12) Patent Application Publication
(10) Pub. No.: US 2017/0165170 A1
(43) Pub. Date: Jun. 15, 2017

(54) EMULSIONS COMPRISING AT LEAST THREE DIFFERENT PRESERVATIVES

(71) Applicant: Henkel AG & Co. KGaA, Duesseldorf (DE)

(72) Inventors: Heike Schelges, Willich (DE); Rainer Simmering, Grevenbroich (DE); Barbara Heide, Krefeld (DE); Melanie Rauschenberg, Kamen (DE)

(73) Assignee: Henkel AG & Co. KGaA, Duesseldorf (DE)

(21) Appl. No.: 15/372,523

(22) Filed: Dec. 8, 2016

(30) Foreign Application Priority Data
Dec. 11, 2015 (DE) 10 2015 225 003.2

(51) Int. Cl. A61K 8/49; A61K 8/06; A61K 8/42; A61K 8/23; A61K 8/92; A61K 8/34; A61K 8/368; A61K 8/43; A61K 8/33; A61Q 19/10; A61K 8/36

(52) U.S. Cl. CPC A61K 8/4953; A61Q 19/10; A61K 8/06; A61K 8/42; A61K 8/4926; A61K 8/23; A61K 8/36; A61K 8/34; A61K 8/361; A61K 8/347; A61K 8/345; A61K 8/368; A61K 8/43; A61K 8/494; A61K 8/33; A61K 8/922; A61K 2800/524; A61K 2800/592

(57) ABSTRACT

Cosmetic agents in the form of emulsions include synergistically effective preservative combinations. The cosmetic agents according to the present invention are used to clean and/or care for skin and/or hair.

### FIELD OF THE INVENTION

The present invention generally relates to cosmetic agents that include at least one emulsifier, at least one oil and/or one wax and/or one ester, at least one special preservative combination, and additionally at least one further preservative. The present invention also relates to the use of such cosmetic agents to clean and care for skin and/or hair.

### BACKGROUND OF THE INVENTION

Due to their composition, cosmetic agents can be a growth medium for germs and microorganisms. These germs can bring about microbial contamination for the consumer on the one hand, and on the other hand can alter the ingredients of the cosmetic, thus forming substances with undesirable effects such as sensitization or skin irritation. These cosmetics must be preserved in order to prevent these undesirable consequences and ensure a certain minimum shelf life thereof. Because preservatives, in turn, have an irritant potential, use thereof in cosmetics is strictly regulated.

The skin microflora has a decisive influence on different cosmetic parameters. Thus, pathogenic germs such as Staphylococcus aureus play a crucial role in the formation of skin blemishes. Recent studies also indicate that an imbalance in the skin microflora can affect the aging of the skin, because undesired germs lead to an increased immune response from the skin, leading in turn to increased inflammatory reactions over the course of which skin aging markers are stimulated.

There therefore has been and continues to be a need for preservative compositions that prevent undesired germs from colonizing the product or the skin, but without interfering or without interfering significantly with the natural skin flora.

The mixture of different antimicrobial substances to increase the antimicrobial activity is generally known. Thus, WO 03/043593 A1 proposes combining conventional antibacterial substances such as triclosan, phenoxyethanol, or hexetidine with ethyl lauroyl arginate in order to intensify the antibacterial effect. WO 2007/014580 A1 proposes preservative mixtures including ethyl lauroyl arginate alongside salts of organic or inorganic acids—in particular, sodium citrate, sodium acetate, sodium glutamate, sodium fumarate, sodium malate, sodium gluconate, sodium laurate, sodium lactate, sodium hexametaphosphate, sodium tert-butylhydroquinate, sodium propylparabenate, or the hydrochlorides of glucosamine or ethanolamine. EP 1414394 B1 discloses cosmetic compositions including a preservative mixture made of ethyl lauroyl arginate and parabens, imidazolidinyl urea, phenoxyethanol, DMDM hydantoin, 2-methyl-5-chloro-3,4-isothiazolinone/2-methyl-3,4-isothiazolinone, and Quaternium-15.

There is therefore also the need to provide antimicrobial compositions that are highly effective when a small amount is used.

In summary, it is desirable to provide cosmetic agents that have a favorable cleaning and caring effect, with simultaneously excellent preservation. In particular, synergistic preservative combinations that are highly effective at low concentrations and enable the production of low-irritant and low-sensitization cosmetic agents due to an overall reduction in amounts used are desirable.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent includes, in a cosmetically acceptable carrier, at least one emulsifier; at least one compound selected from the group consisting of oils, waxes, esters, or mixtures thereof; at least one preservative mixture selected from the group consisting of chloroxylenol and phenoxyisopropanol, undecylenic acid and formic acid, phenoxyisopropanol and piroctone olamine, phenoxyisopropanol and formic acid, sulfite(s) and hexetidine, ethyl lauroyl arginate and formic acid, ethyl lauroyl arginate and chloroxylenol, hexetidine and benzyl alcohol, hexetidine and chloroxylenol, hexetidine and piroctone olamine, hexetidine and chlorophenesin, hexetidine and formic acid, and mixtures thereof, and at least one further preservative selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof, and mixtures of these preservatives.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been surprisingly found that the use of certain preservative mixtures in cosmetic agents leads to a synergistic effect on the preservative action. Therefore, the amount of preservatives used can be reduced without negatively affecting the preservative action. Due to the reduced amount of preservatives, the cosmetic agents according to the present invention are low-irritant and low-sensitization.

The subject matter of the present invention is a cosmetic agent including, in a cosmetically acceptable carrier:
a) at least one emulsifier;
b) at least one compound selected from the group consisting of oils, waxes, esters, or mixtures thereof;
c) at least one preservative mixture selected from the group consisting of
   chloroxylenol and phenoxyisopropanol,
   undecylenic acid and formic acid,
   phenoxyisopropanol and piroctone olamine,
   phenoxyisopropanol and formic acid,
   sulfite(s) and hexetidine,
   ethyl lauroyl arginate and formic acid,
   ethyl lauroyl arginate and chloroxylenol,
   hexetidine and benzyl alcohol,
   hexetidine and chloroxylenol,
   hexetidine and piroctone olamine,
   hexetidine and chlorophenesin,
   hexetidine and formic acid,

and mixtures thereof, and
d) at least one further preservative selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof, and mixtures of these preservatives.

The cosmetic agents of the present invention are preferably emulsions in the form of cleansing milks, impregnation solutions for cleaning cloths, and emulsions for removing eye makeup.

According to the present invention, the term “preservative mixture” is understood to mean a mixture made of two of the preservatives listed above under the feature c).

Furthermore, according to the present invention, the term “emulsifiers” is understood to mean amphiphilic (bifunctional) compounds composed of at least one hydrophobic moiety and at least one hydrophilic moiety. The hydrophobic residue is preferably a hydrocarbon chain having eight to 28 carbon atoms, which may be saturated or unsaturated and linear or branched. Especially preferably, this C8-C28 alkyl chain is linear.

In addition, the term “wax” in the context of the present invention is understood to mean substances that are kneadable or solid to brittle at 20° C., have a coarse to finely crystalline structure, and visually are translucent to opaque but not glassy. Moreover, these substances melt above 25° C. without decomposing, are slightly liquid (slightly viscous) at slightly above the melting point, have a strongly temperature-dependent consistency and solubility, and can be polished under slight pressure.

In addition, according to the present invention, the term “ester” is understood to mean carboxylic acid derivatives having at least one functional group R1-C(O)—O—R2, wherein R1 and R2—each independently of one another—denote C2-C30 alkyl groups, C2-C30 alkylene groups, and C2-C30 aralkyl groups. Preferred esters include exactly one of the aforementioned functional groups. Such esters can be obtained, for example, through the reaction of a carboxylic acid with an alcohol.

Values indicated by wt % presently designate—unless otherwise specified—the total weight of the cosmetic agents according to the present invention, wherein the sum of all ingredients of the agents according to the present invention gives 100 wt %.

As a first essential component a), the cosmetic agent according to the present invention includes at least one emulsifier.

In the context of the present invention, it has proven preferable when the cosmetic agent includes a nonionic emulsifier. Nonionic emulsifiers are understood according to the present invention to mean emulsifiers having no charged groups. Charged groups are understood to mean both permanently cationic and anionic groups and temporarily cationic and anionic groups. Permanently cationic and anionic groups have a cationic or anionic charge irrespective of the pH value. Temporarily cationic and anionic groups, on the other hand, have a cationic or anionic charge only at certain pH values. Preferred cosmetic agents of the present invention are therefore characterized by including at least one emulsifier selected from the group consisting of: (i) addition products of 4 to 30 mol ethylene oxide and/or 1 to 5 mol propylene oxide with linear C8-C22 alcohols, with C12-C22 carboxylic acids, and with C8-C15 alkylphenols; (ii) C12-C22 carboxylic acid mono- and diesters of addition products of 1 to 30 mol ethylene oxide with C3-C6 polyols; (iii) ethylene oxide and polyglycerol addition products with methyl glucoside carboxylic acid esters, carboxylic acid alkanolamides, and carboxylic acid glucamides, C8-C22 alkylmono- and oligoglycosides; (iv) addition products of 5 to 60 mol ethylene oxide with castor oil and hydrogenated castor oil; (v) partial esters of polyols having three to six carbon atoms with saturated C8-C22 carboxylic acids; (vi) sterols; (vii) carboxylic acid esters of sugars and sugar alcohols; and (viii) mixtures thereof.

Advantageously, the at least one emulsifier in the cosmetic agents according to the present invention is used in certain ranges. Preferred cosmetic agents of the present invention are therefore characterized by including—based on the total weight thereof—0.1 to 40 wt %, preferably 0.3 to 35 wt %, preferably 0.5 to 30 wt %, in particular, 1.0 to 20 wt % at least one emulsifier. Use of the aforementioned amounts ensures sufficient emulsification of the ingredients and thus enables high storage stability of the cosmetic agents according to the present invention.

As a second essential component b), the cosmetic agents according to the present invention include at least one compound selected from the group consisting of oils, waxes, esters, or mixtures thereof.

It has proven advantageous in the context of the present invention when the cosmetic agents include at least one volatile non-silicone oil and/or a vegetable oil. Volatile non-silicone oils are understood according to the present invention to mean oils that include no silicon atoms and—at 20° C. and an ambient pressure of 1.013 hPa—have a vapor pressure of 2.66 Pa to 40,000 Pa (0.02 to 300 mmHg), preferably 10 to 12,000 Pa (0.1 to 90 mmHg), further preferably 13 to 3,000 Pa (0.1 to 23 mmHg), in particular, 15 to 500 Pa (0.1 to 4 mmHg). It is therefore preferred according to the present invention when the cosmetic agent includes at least one oil, wherein the oil is selected from the group consisting of: (i) volatile non-silicone oils, in particular, liquid paraffin oils and isoparaffin oils, such as isodecane, isoundecane, isododecane, isotridecane, isotetradecane, isopentadecane, isohexadecane, and isoeicosane; (ii) vegetable oils, in particular, sunflower oil, olive oil, soybean oil, rapeseed oil, almond oil, jojoba oil, orange oil, wheat germ oil, peach kernel oil, and the liquid components of coconut oil; and (iii) mixtures thereof. The use of the aforementioned oils in the cosmetic agents according to the present invention leads to a high care effect and conditioning of the skin and/or hair.

It is furthermore preferably in the context of the present invention when the cosmetic agents include at least one wax. Preferred cosmetic agents of the present invention are therefore characterized by including at least one wax, the wax being selected from the group consisting of: (i); coconut fatty acid glycerol mono-, di-, and triesters; (ii) Butyrospermum parki (Shea butter); (iii) esters of saturated monohydric C8-C18 alcohols with saturated C12-C18 monocarboxylic acids; (iv) linear primary C12-C24 alkanols; (v) esters from a saturated monohydric C16-C60 alkanol and a saturated C8-C36 monocarboxylic acid, in particular, cetyl behenate, stearyl behenate, and C20-C40 alkyl stearate; (vi) glycerol triesters of saturated linear C12-C30 carboxylic acids, which can be hydroxylated, in particular, hydrogenated palm oil, hydrogenated coconut oil, hydrogenated castor oil, glyceryl tribehenate, and glyceryl tri-12-hydroxystearate; (vii) natural vegetable waxes, in particular, candelilla wax, carnauba wax, Japan wax, sugar cane wax, ouricoury wax, cork wax, sunflower wax, fruit waxes; (viii) animal waxes, in particular, bee wax, shellac wax, and spermaceti; (ix) synthetic waxes, in particular montan ester waxes, hydrogenated jojoba waxes and sasol waxes, polyalkylene waxes and polyethylene glycol waxes, C20-C40 dialkyl esters of dimer acids, C30-50 alkyl beeswax, and alkyl and alkyl aryl esters of dimeric fatty acids, paraffin waxes; and (x) mixtures thereof. Commercial products bearing the INCI name Cocoglycerides, in particular the commercial products Novata® (from BASF), particularly preferably Novata® AB, a mixture of C12-18 mono-, di-, and triglycerides that melts in the range from 30 to 32° C., and the products of the Softisan series (Sasol Germany GmbH) bearing the INCI name Hydrogenated Cocoglycerides, in particular Softisan 100, 133, 134, 138, 142, are particularly preferred. Further preferred esters of saturated, monohydric C12-18 alcohols with saturated C12-18 monocarboxylic acids are stearyl laurate, cetearyl stearate (such as Crodamol® CSS), cetyl palmitate (such as Cutina® CP), and myristyl myristate (such as Cetiol® MM). Furthermore, a C20-C40 alkyl stearate is preferably used as the wax component. This ester is known under the name Kester Wax® K82H or Kester Wax® K80H and is sold by Koster Keunen Inc.

In the context of the present invention, it has also proven preferable when the cosmetic agent includes at least one ester. It is therefore preferred according to the present invention when the cosmetic agent includes at least one ester, wherein the ester is selected from the group consisting of: (i) triethyl citrates; (ii) dicarboxylic acid esters of linear or branched C2-C10 alkanols; (iii) symmetric, asymmetric, or cyclic esters of carbonic acid with alcohols; (iv) esters of dimers of unsaturated C12-22 carboxylic acids with monohydric, linear, branched, and cyclic C2-18 alkanols or C2-6 alkanols; (v) benzoic acid esters of linear or branched C8-22 alkanols, such as benzoic acid C12-15 alkyl esters, benzoic acid isostearyl esters, and benzoic acid octyldodecyl esters; and (vi) mixtures thereof. The use of the aforementioned esters also leads to favorable care and conditioning of the skin and/or hair.

Especially preferred embodiments of the present invention include at least one aforementioned oil and/or wax and/or one aforementioned ester.

As a third essential component c), the cosmetic agent includes at least one certain preservative mixture. These preservative mixtures have a synergistic effect in connection with the additional preservative d) on the antimicrobial action, and therefore lead to especially effective preservation of the cosmetic agents according to the present invention. Furthermore, due to the synergistic effect, the amount used thereof can be reduced, so as to yield low-irritant and low-sensitization cosmetic agents.

The cosmetic agents according to the present invention preferably have a certain weight ratio of the preservatives c) included in these agents. It is therefore preferred in the context of the present invention when the cosmetic agents have a weight ratio of the first preservative to the second preservative in the preservative mixture c) of 10:1 to 1:10, preferably 8:1 to 1:8, preferably 5:1 to 1:5, in particular, 2:1 to 1:2. The use of such weight ratios has proven especially advantageous for the synergistic increase in the preservative power of this mixture in combination with the additional preservative d).

The cosmetic agent according to the present invention includes the preservative mixture c) preferably in certain ranges. Preferred cosmetic agents according to the present invention are therefore characterized by including—based on the total weight thereof—0.001 to 10 wt %, preferably 0.005 to 7.0 wt %, preferably 0.01 to 4.0 wt %, in particular, 0.05 to 2.0 wt % at least one preservative mixture (c). The aforementioned amounts refer to the total amount of the preservative mixture, i.e., the aforementioned two preservatives. The use of such amounts of the preservative mixture leads to excellent preservation of the cosmetic agents according to the present invention. Furthermore, due to the synergistic action of the preservative mixture in connection with the at least one further preservative, the amount of preservatives used can be reduced without negatively affecting the preservative power. The cosmetic agents according to the present invention are therefore especially low-irritant and low-sensitization.

As a fourth essential component, the cosmetic agents according to the present invention include at least one additional preservative d) selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof, and mixtures of these preservatives. The addition of this at least one further preservative leads to a synergistic increase in the preservative power, in connection with the aforementioned preservative combination.

It may, however, be preferred in the context of the present invention when a mixture of the aforementioned compounds is used as the preservative d). Preferred cosmetic agents according to the present invention are therefore characterized by including at least two further preservatives d) selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof.

Further preferred cosmetic agents according to the present invention are characterized by including at least three further preservatives d) selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof.

Moreover, cosmetic agents that are advantageous according to the present invention are those including at least four further preservatives d) selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof.

Finally, cosmetic agents according to the present invention are preferably those including a mixture made of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, and citric acid as further preservatives d).

Particularly preferably, the cosmetic agents according to the present invention include the following preservatives or mixtures of these preservatives KM1 to KM466, as the additional preservative d) (where be=sodium benzoate, pr=propionic acid, sa=sodium salicylate, zn=zinc pyrithione, pb=paraben, pg=propyl aminopropyl biguanide, pe=phenoxyethanol, cl=climbazole, chi=chlorhexidine, be=behentrimonium chloride, cb=cetrimonium bromide, cc=cetrimonium chloride, lb=laurtrimonium bromide, lc=laurtrimonium chloride, sb=steartrimonium bromide, sc=steartrimonium chloride, gl=glutaraldehyde, zi=citric acid, siz=silver citrate).

| KM1 | KM2 | KM3 | KM4 | KM5 | KM6 | KM7 | KM8 |
|---|---|---|---|---|---|---|---|
| be | pr | sa | zn | pb | pg | pe | Cl |
| KM9 | KM10 | KM11 | KM12 | KM13 | KM14 | KM15 | KM16 |
| chi | bc | cb | cc | lb | lc | sb | Sc |
| KM17 | KM18 | KM19 | KM20 | KM21 | KM22 | KM23 | KM24 |
| gl | zi | siz | be + pr | be + pr + sa | be + pr + zn | be + pr + pb | be + pr + pg |
| KM25 | KM26 | KM27 | KM28 | KM29 | KM30 | KM31 | KM32 |
| be + pr + pe | be + pr + cl | be + pr + chi | be + pr + bc | be + pr + cb | be + pr + cc | be + pr + lb | be + pr + lc |
| KM33 | KM34 | KM35 | KM36 | KM37 | KM38 | KM39 | KM40 |
| be + pr + sb | be + pr + sc | be + pr + gl | be + pr + zi | be + pr + siz | be + sa + zn | be + sa + | be + sa + pg |
| | | | | | | pb¹⁾ | |
| KM41 | KM42 | KM43 | KM44 | KM45 | KM46 | KM47 | KM48 |
| be + sa + pe | be + sa + cl | be + sa + chi | be + sa + bc | be + sa + cb | be + sa + cc | be + sa + lb | be + sa + lc |
| KM49 | KM50 | KM51 | KM52 | KM53 | KM54 | KM55 | KM56 |
| be + sa + sb | be + sa + sc | be + sa + gl | be + sa + zi | be + sa + siz | be + zn + | be + zn + pg | be + zn + pe |
| | | | | | pb¹⁾ | | |
| KM57 | KM58 | KM59 | KM60 | KM61 | KM62 | KM63 | KM64 |
| be + zn + cl | be + zn + | be + zn + bc | be + zn + cb | be + zn + cc | be + zn + lb | be + zn + lc | be + zn + sb |
| | chi | | | | | | |
| KM65 | KM66 | KM67 | KM68 | KM69 | KM70 | KM71 | KM72 |
| be + zn + sc | be + zn + gl | be + zn + zi | be + zn + siz | be + pb¹⁾ + | be + pb¹⁾ + | be + pb + cl | be + pb¹⁾ + |
| | | | | pg | pe | | chi |
| KM73 | KM74 | KM75 | KM76 | KM77 | KM78 | KM79 | KM80 |
| be + pb¹⁾ + | be + pb¹⁾ + | be + pb¹⁾ + | be + pb¹⁾ + | be + pb¹⁾ + | be + pb¹⁾ + | be + pb¹⁾ + | be + pb¹⁾ + |
| bc | cb | cc | lb | lc | sb | sc | gl |
| KM81 | KM82 | KM83 | KM84 | KM85 | KM86 | KM87 | KM88 |
| be + pb¹⁾ + | be + pb¹⁾ + | be + pg + pe | be + pg + cl | be + pg + chi | be + pg + bc | be + pg + cb | be + pg + cc |
| zi | siz | | | | | | |
| KM89 | KM90 | KM91 | KM92 | KM93 | KM94 | KM95 | KM96 |
| be + pg + lb | be + pg + lc | be + pg + sb | be + pg + sc | be + pg + gl | be + pg + zi | be + pg + siz | be + pe + cl |
| KM97 | KM98 | KM99 | KM100 | KM101 | KM102 | KM103 | KM104 |
| be + pe + chi | be + pe + bc | be + pe + cb | be + pe + cc | be + pe + lb | be + pe + lc | be + pe + sb | be + pe + sc |
| KM105 | KM106 | KM107 | KM108 | KM109 | KM110 | KM111 | KM112 |
| be + pe + gl | be + pe + zi | be + pe + siz | be + cl + chi | be + cl + bc | be + cl + cb | be + cl + cc | be + cl + lb |
| KM113 | KM114 | KM115 | KM116 | KM117 | KM118 | KM119 | KM120 |
| be + cl + lc | be + cl + sb | be + cl + sc | be + cl + gl | be + cl + zi | be + cl + siz | be + chi + | be + chi + cb |
| | | | | | | bc | |
| KM121 | KM122 | KM123 | KM124 | KM125 | KM126 | KM127 | KM128 |
| be + bc + siz | be + cb + cc | be + cb + lb | be + cb + lc | be + cb + sb | be + cb + sc | be + cb + gl | be + cb + zi |
| KM129 | KM130 | KM131 | KM132 | KM133 | KM134 | KM135 | KM136 |
| be + lc + gl | be + lc + zi | be + lc + siz | be + sb + sc | be + sb + gl | be + sb + zi | be + sb + | be + sc + gl |
| | | | | | | siz | |
| KM137 | KM138 | KM139 | KM140 | KM141 | KM142 | KM143 | KM144 |
| pr + sa + lc | pr + sa + sb | pr + sa + sc | pr + sa + gl | pr + sa + zi | pr + sa + siz | pr + zn + | pr + zn + pg |
| | | | | | | pb¹⁾ | |
| KM145 | KM146 | KM147 | KM148 | KM149 | KM150 | KM151 | KM152 |
| pr + pb¹⁾ + | pr + pb¹⁾ + | pr + pb¹⁾ + | pr + pb¹⁾ + | pr + pb¹⁾ + | pr + pb¹⁾ + | pr + pb¹⁾ + | pr + pb¹⁾ + |
| chi | bc | cb | cc | lb | lc | sb | sc |
| KM153 | KM154 | KM155 | KM156 | KM157 | KM158 | KM159 | KM160 |
| pr + pe + cl | pr + pe + chi | pr + pe + bc | pr + pe + cb | pr + pe + cc | pr + pe + lb | pr + pe + lc | pr + pe + sb |
| KM161 | KM162 | KM163 | KM164 | KM165 | KM166 | KM167 | KM168 |
| pr + chi + cb | pr + chi + cc | pr + chi + lb | pr + chi + lc | pr + chi + sb | pr + chi + sc | pr + chi + gl | pr + chi + zi |
| KM169 | KM170 | KM171 | KM172 | KM173 | KM174 | KM175 | KM176 |
| pr + cb + zi | pr + cb + siz | pr + cc + lb | pr + cc + lc | pr + cc + sb | pr + cc + sc | pr + cc + gl | pr + cc + zi |
| KM177 | KM178 | KM179 | KM180 | KM181 | KM182 | KM183 | KM184 |
| pr + sc + gl | pr + sc + zi | pr + sc + siz | pr + gl + zi | pr + gl + siz | pr + zi + siz | be + sa | be + zn |
| KM185 | KM186 | KM187 | KM188 | KM189 | KM190 | KM191 | KM192 |
| pr + pg | pr + pg | pr + pe | pr + cl | pr + chi | pr + bc | pr + cb | pr + cc |
| KM193 | KM194 | KM195 | KM196 | KM197 | KM198 | KM199 | KM200 |
| sa + lb | sa + lc | sa + sb | sa + gl | sa + zi | sa + siz | sa + zn + pb | sa + zn + pg |
| KM201 | KM202 | KM203 | KM204 | KM205 | KM206 | KM207 | KM208 |
| sa + pb¹⁾ + | sa + pb¹⁾ + | sa + pb¹⁾ + | sa + pb¹⁾ + | sa + pb¹⁾ + | sa + pb¹⁾ + | sa + pb¹⁾ + | sa + pb¹⁾ + |
| chi | bc | cb | cc | lb | lc | sb | sc |
| KM209 | KM210 | KM211 | KM212 | KM213 | KM214 | KM215 | KM216 |
| sa + pe + cl | sa + pe + chi | sa + pe + bc | sa + pe + cb | sa + pe + cc | sa + pe + lb | sa + pe + lc | sa + pe + sb |
| KM217 | KM218 | KM219 | KM220 | KM221 | KM222 | KM223 | KM224 |
| sa + chi + cb | sa + chi + cc | sa + chi + lb | sa + chi + lc | sa + chi + sb | sa + chi + sc | sa + chi + gl | sa + chi + zi |
| KM225 | KM226 | KM227 | KM228 | KM229 | KM230 | KM231 | KM232 |
| sa + cb + zi | sa + cb + siz | sa + cc + lb | sa + cc + lc | sa + cc + sb | sa + cc + sc | sa + cc + gl | sa + cc + zi |
| KM233 | KM234 | KM235 | KM236 | KM237 | KM238 | KM239 | KM240 |
| sa + sc + gl | sa + sc + zi | sa + sc + siz | sa + gl + zi | sa + gl + siz | sa + zi + siz | zn + pb | zn + pg |
| KM241 | KM242 | KM243 | KM244 | KM245 | KM246 | KM247 | KM248 |
| zn + pb¹⁾ + | zn + pb¹⁾ + | zn + pb¹⁾ + | zn + pb¹⁾ + | zn + pb¹⁾ + | zn + pb¹⁾ + | zn + pb¹⁾ + | zn + pb¹⁾ + |
| bc | cb | cc | lb | lc | sb | sc | gl |
| KM249 | KM250 | KM251 | KM252 | KM253 | KM254 | KM255 | KM256 |
| zn + pe + chi | zn + pe + bc | zn + pe + cb | zn + pe + cc | zn + pe + lb | zn + pe + lc | zn + pe + sb | zn + pe + sc |
| KM257 | KM258 | KM259 | KM260 | KM261 | KM262 | KM263 | KM264 |
| zn + chi + cc | zn + chi + lb | zn + chi + lc | zn + chi + sb | zn + chi + sc | zn + chi + gl | zn + chi + zi | zn + chi + |
| | | | | | | | siz |
| KM265 | KM266 | KM267 | KM268 | KM269 | KM270 | KM271 | KM272 |
| zn + cb + siz | zn + cc + lb | zn + cc + lc | zn + cc + sb | zn + cc + sc | zn + cc + gl | zn + cc + zi | zn + cc + siz |
| KM273 | KM274 | KM275 | KM276 | KM277 | KM278 | KM279 | KM280 |
| zn + sc + zi | zn + sc + siz | zn + gl + zi | zn + gl + siz | zn + zi + siz | pb¹⁾ + pg | pb¹⁾ + pe | pb¹⁾ + cl |
| KM281 | KM282 | KM283 | KM284 | KM285 | KM286 | KM287 | KM288 |
| pb¹⁾ + cc | pb¹⁾ + lb | pb¹⁾ + lc | pb¹⁾ + sb | pb¹⁾ + sc | pb¹⁾ + gl | pb¹⁾ + zi | pb¹⁾ + siz |
| KM289 | KM290 | KM291 | KM292 | KM293 | KM294 | KM295 | KM296 |
| pb¹⁾ + | pb¹⁾ + | pb¹⁾ + | pb¹⁾ + | pb¹⁾ + | pb¹⁾ + | pb¹⁾ + | pb¹⁾ + |
| pe + cb | pe + cc | pe + lb | pe + lc | pe + sb | pe + sc | pe + gl | pe + zi |
| KM297 | KM298 | KM299 | KM300 | KM301 | KM302 | KM303 | KM304 |
| pb¹⁾ + | pb¹⁾ + | pb¹⁾ + | pb¹⁾ + | pb¹⁾ + | pb¹⁾ + | pb¹⁾ + | pb¹⁾ + |
| chi + lc | chi + sb | chi + sc | chi + gl | chi + zi | chi + siz | bc + cb | bc + cc |
| KM305 | KM306 | KM307 | KM308 | KM309 | KM310 | KM311 | KM312 |
| pb¹⁾ + | pb¹⁾ + | pb¹⁾ + | pb¹⁾ + | pb¹⁾ + | pb¹⁾ + | pb¹⁾ + | pb¹⁾ + |
| cc + lc | cc + sb | cc + sc | cc + gl | cc + zi | cc + siz | lb + lc | lb + sb |
| KM313 | KM314 | KM315 | KM316 | KM317 | KM318 | KM319 | KM320 |
| pb¹⁾ + gl + zi | pb¹⁾ + gl + | pb¹⁾ + zi + | pg + pe | pg + cl | pg + chi | pg + bc | pg + cb |
| | siz | siz | | | | | |
| KM321 | KM322 | KM323 | KM324 | KM325 | KM326 | KM327 | KM328 |
| pg + pe + gl | pg + pe + zi | pg + pe + siz | pg + cl + chi | pg + cl + bc | pg + cl + cb | pg + cl + cc | pg + cl + lb |
| KM329 | KM330 | KM331 | KM332 | KM333 | KM334 | KM335 | KM336 |
| pg + bc + cb | pg + bc + cc | pg + bc + lb | pg + bc + lc | pg + bc + sb | pg + bc + sc | pg + bc + gl | pg + bc + zi |
| KM337 | KM338 | KM339 | KM340 | KM341 | KM342 | KM343 | KM344 |
| pg + lb + lc | pg + lb + sb | pg + lb + sc | pg + lb + gl | pg + lb + zi | pg + lb + siz | pg + lc + sb | pg + lc + sc |
| KM345 | KM346 | KM347 | KM348 | KM349 | KM350 | KM351 | KM352 |
| pe + cb | pe + cc | pe + lb | pe + lc | pe + sb | pe + gl | pe + zi | pe + siz |
| KM353 | KM354 | KM355 | KM356 | KM357 | KM358 | KM359 | KM360 |
| pe + chi + sb | pe + chi + | pe + chi + gl | pe + chi + zi | pe + chi + | pe + bc + cb | pe + bc + cc | pe + bc + lb |
| | sc | | | siz | | | |
| KM361 | KM362 | KM363 | KM364 | KM365 | KM366 | KM367 | KM368 |
| pe + cc + sb | pe + cc + sc | pe + cc + gl | pe + cc + zi | pe + cc + siz | pe + lb + lc | pe + lb + sb | pe + lb + sc |
| KM369 | KM370 | KM371 | KM372 | KM373 | KM374 | KM375 | KM376 |
| pe + gl + siz | pe + zi + siz | cl + chi | cl + bc | cl + cb | cl + cc | cl + lb | cl + lc |
| KM377 | KM378 | KM379 | KM380 | KM381 | KM382 | KM383 | KM384 |
| cl + bc + lb | cl + bc + lc | cl + bc + sb | cl + bc + sc | cl + bc + gl | cl + bc + zi | cl + bc + siz | cl + cb + cc |
| KM385 | KM386 | KM387 | KM388 | KM389 | KM390 | KM391 | KM392 |
| cl + lb + sc | cl + lb + gl | cl + lb + zi | cl + lb + siz | cl + lc + sb | cl + lc + sc | cl + lc + gl | cl + lc + zi |
| KM393 | KM394 | KM395 | KM396 | KM397 | KM398 | KM399 | KM400 |
| chi + sb | chi + gl | chi + zi | chi + siz | chi + bc + cb | chi + bc + cc | chi + bc + lb | chi + bc + lb |
| KM401 | KM402 | KM403 | KM404 | KM405 | KM406 | KM407 | KM408 |
| chi + cc + sc | chi + cc + gl | chi + cc + zi | chi + cc + siz | chi + lb + lc | chi + lb + sb | chi + lb + sc | chi + lb + gl |
| KM409 | KM410 | KM411 | KM412 | KM413 | KM414 | KM415 | KM416 |
| chi + zi + siz | bc + cb | bc + cc | bc + lb | bc + lc | bc + sb | bc + gl | bc + zi |
| KM417 | KM418 | KM419 | KM420 | KM421 | KM422 | KM423 | KM424 |
| bc + lb + lc | bc + lb + sb | bc + lb + sc | bc + lb + gl | bc + lb + zi | bc + lb + siz | bc + lc + sb | bc + lc + sc |
| KM425 | KM426 | KM427 | KM428 | KM429 | KM430 | KM431 | KM432 |
| cb + sb | cb + gl | cb + zi | cb + siz | cb + lb + lc | cb + lb + sb | cb + lb + sc | cb + lb + gl |
| KM433 | KM434 | KM435 | KM436 | KM437 | KM438 | KM439 | KM440 |
| cb + zi + siz | lb + lc | lb + sb | lb + gl | lb + zi | lb + siz | lb + lc + sb | lb + lc + sc |
| KM441 | KM442 | KM443 | KM444 | KM445 | KM446 | KM447 | KM448 |
| lc + siz | lc + sb + sc | lc + sb + gl | lc + sb + zi | lc + sb + siz | lc + sc + gl | lc + sc + zi | lc + sc + siz |
| KM449 | KM450 | KM451 | KM452 | KM453 | KM454 | KM455 | KM456 |
| cb + cc + lb | cb + cc + lc | cb + cc + sb | cb + cc + sc | cb + cc + gl | cb + cc + zi | cb + cc + siz | cc + lb |
| KM457 | KM458 | KM459 | KM460 | KM461 | KM462 | KM463 | KM464 |
| cc + sb + sc | cc + sb + gl | cc + sb + zi | cc + sb + siz | cc + sc + gl | cc + sc + zi | cc + sc + siz | cc + gl + zi |

| KM465 | KM466 |
|---|---|
| cc + gl + siz | cc + zi + siz |

¹⁾Paraben is selected from the group consisting of methylparaben, ethylparaben, propylparaben, butylparaben, and mixtures thereof

In this context, it is advantageous when the at least one additional preservative d) is used in a certain total amount. It is therefore preferred according to the present invention when the cosmetic agents include—based on the total weight thereof—0.001 to 10 wt %, preferably 0.005 to 9.0 wt %, preferably 0.05 to 8.0 wt %, in particular, 0.1 to 7.0 wt % at least one further preservative d). If more than one additional preservative d) is used, the total amounts given above indicate the mixture of these preservatives. The use of such amounts of the at least one additional preservative leads to a synergistic increase in the preservative power, in combination with the at least one preservative mixture c).

The following table sets forth preferred embodiments AF1 to AF505 of the cosmetic agents according to the present invention (all values represent wt %). Here, c denotes chloroxylenol, p denotes phenoxyisopropanol, u denotes undecylenic acid, a denotes formic acid, pi denotes piroctone olamine, s denotes sulfite, h denotes hexetidine, e denotes ethyl lauroyl arginate * HCl, ch denotes chlorphenesin, and b denotes benzyl alcohol. Also, in the table, the indication (c+p) (1:2) denotes a mixture made of chloroxylenol and phenoxyisopropanol at a weight ratio 1:2 (based on the total weight of the mixture). (c+p)+(u+a) denotes the combination of a mixture made of chloroxylenol and phenoxyisopropanol with a mixture made of undecylenic acid and formic acid. The aforementioned preservative or preservative mixtures KM1 to KM466 are used, respectively in each case, as the additional preservative d) (marked as “KM” in the table).

| | AF1 | | AF2 | | AF3 |
|---|---|---|---|---|---|
| Emulsifier | 1.0-20 | Emulsifier | 1.0-20 | Emulsifier | 1.0-20 |
| Oil | 1.0-30 | Oil | 1.0-30 | Oil | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF4 | | AF5 | | AF6 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (c + p) + (p + a) | 0.05-2 | (c + p) + (s²⁾ + h) | 0.05-2 | (c + p) + (e + a) | 0.05-2 |
| KM | 0.1-7.0 | KM | 0.1-7.0 | KM | 0.1-7.0 |
| | AF7 | | AF8 | | AF9 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF10 | | AF11 | | AF12 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF13 | | AF14 | | AF15 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF16 | | AF17 | | AF18 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (c + p) + (p + a) | 0.05-2 | (c + p) + (s²⁾ + h) | 0.05-2 | (c + p) + (e + a) | 0.05-2 |
| KM | 0.1-7.0 | KM | 0.1-7.0 | KM | 0.1-7.0 |
| | AF19 | | AF20 | | AF21 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF22 | | AF23 | | AF24 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF25 | | AF26 | | AF27 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF28 | | AF29 | | AF30 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (c + p) + (p + a) | 0.05-2 | (c + p) + (s²⁾ + h) | 0.05-2 | (c + p) + (e + a) | 0.05-2 |
| KM | 0.1-7.0 | KM | 0.1-7.0 | KM | 0.1-7.0 |
| | AF31 | | AF32 | | AF33 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF34 | | AF35 | | AF36 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF37 | | AF38 | | AF39 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (u + a) (1:3) | 0.05-2 | (u + a) + (p + pi) | 0.05-2 | (u + a) + (p + a) | 0.05-2 |
| KM | 0.1-7.0 | KM | 0.1-7.0 | KM | 0.1-7.0 |
| | AF40 | | AF41 | | AF42 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (u + a) + ( s²⁾ + h) | 0.05-2 | (u + a) + (e + a) | 0.05-2 | (u + a) + (e + c) | 0.05-2 |
| KM | 0.1-7.0 | KM | 0.1-7.0 | KM | 0.1-7.0 |
| | AF43 | | AF44 | | AF45 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (u + a) + (h + b) | 0.05-2 | (u + a) + (h + c) | 0.05-2 | (u + a) + (h + pi) | 0.05-2 |
| KM | 0.1-7.0 | KM | 0.1-7.0 | KM | 0.1-7.0 |
| | AF46 | | AF47 | | AF48 |
| Emulsifier | 1.0-20 | Emulsifier | 1.0-20 | Emulsifier | 1.0-20 |
| Oil | 1.0-30 | Oil | 1.0-30 | Wax | 1.0-30 |
| (u + a) + (h + ch) | 0.05-2 | (u + a) + (h + a) | 0.05-2 | (u + a) (1:3) | 0.05-2 |
| KM | 0.1-7.0 | KM | 0.1-7.0 | KM | 0.1-7.0 |
| | AF49 | | AF50 | | AF51 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (u + a) + (p + pi) | 0.05-2 | (u + a) + (p + a) | 0.05-2 | (u + a) + ( s²⁾ + h) | 0.05-2 |
| KM | 0.1-7.0 | KM | 0.1-7.0 | KM | 0.1-7.0 |
| | AF52 | | AF53 | | AF54 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (u + a) + (e + a) | 0.05-2 | (u + a) + (e + c) | 0.05-2 | (u + a) + (h + b) | 0.05-2 |
| KM | 0.1-7.0 | KM | 0.1-7.0 | KM | 0.1-7.0 |
| | AF55 | | AF59 | | AF57 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (u + a) + (h + c) | 0.05-2 | (u + a) + (h + pi) | 0.05-2 | (u + a) + (h + ch) | 0.05-2 |
| KM | 0.1-7.0 | KM | 0.1-7.0 | KM | 0.1-7.0 |
| | AF58 | | AF59 | | AF60 |
| Emulsifier | 1.0-20 | Emulsifier | 1.0-20 | Emulsifier | 1.0-20 |
| Wax | 1.0-30 | Ester | 1.0-30 | Ester | 1.0-30 |
| (u + a) + (h + a) | 0.05-2 | (u + a) (1:3) | 0.05-2 | (u + a) + (p + pi) | 0.05-2 |
| KM | 0.1-7.0 | KM | 0.1-7.0 | KM | 0.1-7.0 |
| | AF61 | | AF62 | | AF63 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (u + a) + (p + a) | 0.05-2 | (u + a) + ( s²⁾ + h) | 0.05-2 | (u + a) + (e + a) | 0.05-2 |
| KM | 0.1-7.0 | KM | 0.1-7.0 | KM | 0.1-7.0 |
| | AF64 | | AF65 | | AF66 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (u + a) + (e + c) | 0.05-2 | (u + a) + (h + b) | 0.05-2 | (u + a) + (h + c) | 0.05-2 |
| KM | 0.1-7.0 | KM | 0.1-7.0 | KM | 0.1-7.0 |
| | AF67 | | AF68 | | AF69 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (u + a) + (h + pi) | 0.05-2 | (u + a) + (h + ch) | 0.05-2 | (u + a) + (h + a) | 0.05-2 |
| KM | 0.1-7.0 | KM | 0.1-7.0 | KM | 0.1-7.0 |
| | AF70 | | AF71 | | AF72 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (p + pi) (1:1) | 0.05-2 | (p + pi) + (p + a) | 0.05-2 | (p + pi) + (s²⁾ + h) | 0.05-2 |
| KM | 0.1-7.0 | KM | 0.1-7.0 | KM | 0.1-7.0 |
| | AF73 | | AF74 | | AF75 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (p + pi) + (e + a) | 0.05-2 | (p + pi) + (e + c) | 0.05-2 | (p + pi) + (h + b) | 0.05-2 |
| KM | 0.1-7.0 | KM | 0.1-7.0 | KM | 0.1-7.0 |
| | AF76 | | AF77 | | AF78 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (p + pi) + (h + c) | 0.05-2 | (p + pi) + (h + pi) | 0.05-2 | (p + pi) + (h + ch) | 0.05-2 |
| KM | 0.1-7.0 | KM | 0.1-7.0 | KM | 0.1-7.0 |
| | AF79 | | AF80 | | AF81 |
| Emulsifier | 1.0-20 | Emulsifier | 1.0-20 | Emulsifier | 1.0-20 |
| Oil | 1.0-30 | Wax | 1.0-30 | Wax | 1.0-30 |
| (p + pi) + (h + a) | 0.05-2 | (p + pi) (1:1) | 0.05-2 | (p + pi) + (p + a) | 0.05-2 |
| KM | 0.1-7.0 | KM | 0.1-7.0 | KM | 0.1-7.0 |
| | AF82 | | AF83 | | AF84 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (p + pi) + (s²⁾ + h) | 0.05-2 | (p + pi) + (e + a) | 0.05-2 | (p + pi) + (e + c) | 0.05-2 |
| KM | 0.1-7.0 | KM | 0.1-7.0 | KM | 0.1-7.0 |
| | AF85 | | AF86 | | AF87 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (p + pi) + (h + b) | 0.05-2 | (p + pi) + (h + c) | 0.05-2 | (p + pi) + (h + pi) | 0.05-2 |
| KM | 0.1-7.0 | KM | 0.1-7.0 | KM | 0.1-7.0 |
| | AF88 | | AF89 | | AF90 |
| Emulsifier | 1.0-20 | Emulsifier | 1.0-20 | Emulsifier | 1.0-20 |
| Wax | 1.0-30 | Ester | 1.0-30 | Ester | 1.0-30 |
| (p + pi) + (h + a) | 0.05-2 | (p + pi) (1:1) | 0.05-2 | (p + pi) + (p + a) | 0.05-2 |
| KM | 0.1-7.0 | KM | 0.1-7.0 | KM | 0.1-7.0 |
| | AF91 | | AF92 | | AF93 |
| Emulsifier | 1.0-20 | Emulsifier | 1.0-20 | Emulsifier | 1.0-20 |
| Wax | 1.0-30 | Ester | 1.0-30 | Ester | 1.0-30 |
| (p + pi) + (s²⁾ + h) | 0.05-2 | (p + pi) + (e + a) | 0.05-2 | (p + pi) + (e + c) | 0.05-2 |
| KM | 0.1-7.0 | KM | 0.1-7.0 | KM | 0.1-7.0 |
| | AF94 | | AF95 | | AF96 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (p + pi) + (h + b) | 0.05-2 | (p + pi) + (h + c) | 0.05-2 | (p + pi) + (h + pi) | 0.05-2 |
| KM | 0.1-7.0 | KM | 0.1-7.0 | KM | 0.1-7.0 |
| | AF97 | | AF98 | | AF99 |
| Emulsifier | 1.0-20 | Emulsifier | 1.0-20 | Emulsifier | 1.0-20 |
| Ester | 1.0-30 | Ester | 1.0-30 | Wax | 1.0-30 |
| (p + pi) + (h + ch) | 0.05-2 | (p + pi) + (h + a) | 0.05-2 | (p + pi) + (h + ch) | 0.05-2 |
| KM | 0.1-7.0 | KM | 0.1-7.0 | KM | 0.1-7.0 |
| | AF100 | | AF101 | | AF102 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (s²⁾ + h) (2:1) | 0.05-2 | (s²⁾ + h) + (e + a) | 0.05-2 | (s²⁾ + h) + (e + c) | 0.05-2 |
| KM | 0.1-7.0 | KM | 0.1-7.0 | KM | 0.1-7.0 |
| | AF103 | | AF104 | | AF105 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (s²⁾ + h) + (h + b) | 0.05-2 | (p + pi) + (e + c) | 0.05-2 | (p + pi) + (h + b) | 0.05-2 |
| KM | 0.1-7.0 | KM | 0.1-7.0 | KM | 0.1-7.0 |
| | AF106 | | AF107 | | AF108 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (s²⁾ + h) + (h + c) | 0.05-2 | (s²⁾ + h) + (h + pi) | 0.05-2 | (s²⁾ + h) + (h + ch) | 0.05-2 |
| KM | 0.1-7.0 | KM | 0.1-7.0 | KM | 0.1-7.0 |
| | AF109 | | AF110 | | AF111 |
| Emulsifier | 1.0-20 | Emulsifier | 1.0-20 | Emulsifier | 1.0-20 |
| Oil | 1.0-30 | Wax | 1.0-30 | Wax | 1.0-30 |
| (s²⁾ + h) + (h + a) | 0.05-2 | (s²⁾ + h) (2:1) | 0.05-2 | (s²⁾ + h) + (e + a) | 0.05-2 |
| KM | 0.1-7.0 | KM | 0.1-7.0 | KM | 0.1-7.0 |
| | AF112 | | AF113 | | AF114 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (s²⁾ + h) + (e + c) | 0.05-2 | (s²⁾ + h) + (h + b) | 0.05-2 | (s²⁾ + h) + (h + c) | 0.05-2 |
| KM | 0.1-7.0 | KM | 0.1-7.0 | KM | 0.1-7.0 |
| | AF115 | | AF116 | | AF117 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (s²⁾ + h) + (h + pi) | 0.05-2 | (s²⁾ + h) + (h + ch) | 0.05-2 | (s²⁾ + h) + (h + a) | 0.05-2 |
| KM | 0.1-7.0 | KM | 0.1-7.0 | KM | 0.1-7.0 |
| | AF118 | | AF119 | | AF120 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (s²⁾ + h) (2:1) | 0.05-2 | (s²⁾ + h) + (e + a) | 0.05-2 | (s²⁾ + h) + (e + c) | 0.05-2 |
| KM | 0.1-7.0 | KM | 0.1-7.0 | KM | 0.1-7.0 |
| | AF121 | | AF122 | | AF123 |
| Emulsifier | 1.0-20 | Emulsifier | 1.0-20 | Emulsifier | 1.0-20 |
| Wax | 1.0-30 | Ester | 1.0-30 | Ester | 1.0-30 |
| (s²⁾ + h) + (h + b) | 0.05-2 | (s²⁾ + h) + (h + c) | 0.05-2 | (s²⁾ + h) + (h + pi) | 0.05-2 |
| KM | 0.1-7.0 | KM | 0.1-7.0 | KM | 0.1-7.0 |
| | AF124 | | AF125 | | AF126 |
| Emulsifier | 1.0-20 | Emulsifier | 1.0-20 | Emulsifier | 1.0-20 |
| Ester | 1.0-30 | Ester | 1.0-30 | Oil | 1.0-30 |
| (s²⁾ + h) + (h + ch) | 0.05-2 | (s²⁾ + h) + (h + a) | 0.05-2 | (e + a) (1:2) | 0.05-2 |
| KM | 0.1-7.0 | KM | 0.1-7.0 | KM | 0.1-7.0 |
| | AF127 | | AF128 | | AF129 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (e + a) + (e + c) | 0.05-2 | (e + a) + (h + b) | 0.05-2 | (e + a) + (h + c) | 0.05-2 |
| KM | 0.1-7.0 | KM | 0.1-7.0 | KM | 0.1-7.0 |
| | AF130 | | AF131 | | AF132 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (e + a) + (h + pi) | 0.05-2 | (e + a) + (h + ch) | 0.05-2 | (e + a) + (h + a) | 0.05-2 |
| KM | 0.1-7.0 | KM | 0.1-7.0 | KM | 0.1-7.0 |
| | AF133 | | AF134 | | AF135 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (e + a) (1:2) | 0.05-2 | (e + a) + (e + c) | 0.05-2 | (e + a) + (h + b) | 0.05-2 |
| KM | 0.1-7.0 | KM | 0.1-7.0 | KM | 0.1-7.0 |
| | AF136 | | AF137 | | AF138 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (e + a) + (h + c) | 0.05-2 | (e + a) + (h + pi) | 0.05-2 | (e + a) + (h + ch) | 0.05-2 |
| KM | 0.1-7.0 | KM | 0.1-7.0 | KM | 0.1-7.0 |
| | AF139 | | AF140 | | AF141 |
| Emulsifier | 1.0-20 | Emulsifier | 1.0-20 | Emulsifier | 1.0-20 |
| Wax | 1.0-30 | Wax | 1.0-30 | Ester | 1.0-30 |
| (e + a) + (h + a) | 0.05-2 | (e + a) + (h + ch) | 0.05-2 | (e + a) (1:2) | 0.05-2 |
| KM | 0.1-7.0 | KM | 0.1-7.0 | KM | 0.1-7.0 |
| | AF139 | | AF140 | | AF141 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (e + a) + (e + c) | 0.05-2 | (e + a) + (h + b) | 0.05-2 | (e + a) + (h + c) | 0.05-2 |
| KM | 0.1-7.0 | KM | 0.1-7.0 | KM | 0.1-7.0 |
| | AF142 | | AF143 | | AF144 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (e + a) + (h + pi) | 0.05-2 | (e + a) + (h + ch) | 0.05-2 | (e + a) + (h + a) | 0.05-2 |
| KM | 0.1-7.0 | KM | 0.1-7.0 | KM | 0.1-7.0 |
| | AF145 | | AF146 | | AF147 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF148 | | AF149 | | AF150 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF151 | | AF152 | | AF153 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF154 | | AF155 | | AF156 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF157 | | AF158 | | AF159 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF160 | | AF161 | | AF162 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF163 | | AF164 | | AF165 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF166 | | AF167 | | AF168 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF169 | | AF170 | | AF171 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF172 | | AF172 | | AF173 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF174 | | AF175 | | AF176 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (h + c) (1:5) | 0.05-2 | (h + c) + (h + pi) | 0.05-2 | (e + c) + (h + ch) | 0.05-2 |
| KM | 0.1-7.0 | KM | 0.1-7.0 | KM | 0.1-7.0 |
| | AF177 | | AF178 | | AF179 |
| Emulsifier | 1.0-20 | Emulsifier | 1.0-20 | Emulsifier | 1.0-20 |
| Oil | 1.0-30 | Wax | 1.0-30 | Wax | 1.0-30 |
| (h + c) + (h + a) | 0.05-2 | (h + c) (1:5) | 0.05-2 | (h + c) + (h + pi) | 0.05-2 |
| KM | 0.1-7.0 | KM | 0.1-7.0 | KM | 0.1-7.0 |
| | AF180 | | AF181 | | AF182 |
| Emulsifier | 1.0-20 | Emulsifier | 1.0-20 | Emulsifier | 1.0-20 |
| Wax | 1.0-30 | Wax | 1.0-30 | Ester | 1.0-30 |
| (h + c) + (h + ch) | 0.05-2 | (h + c) + (h + a) | 0.05-2 | (h + c) (1:5) | 0.05-2 |
| KM | 0.1-7.0 | KM | 0.1-7.0 | KM | 0.1-7.0 |
| | AF183 | | AF184 | | AF185 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (h + c) + (h + pi) | 0.05-2 | (h + c) + (h + ch) | 0.05-2 | (h + c) + (h + a) | 0.05-2 |
| KM | 0.1-7.0 | KM | 0.1-7.0 | KM | 0.1-7.0 |
| | AF186 | | AF187 | | AF188 |
| Emulsifier | 1.0-20 | Emulsifier | 1.0-20 | Emulsifier | 1.0-20 |
| Oil | 1.0-30 | Oil | 1.0-30 | Oil | 1.0-30 |
| (h + pi) (1:10) | 0.05-2 | (h + pi) + (h + ch) | 0.05-2 | (h + pi) + (h + a) | 0.05-2 |
| KM | 0.1-7.0 | KM | 0.1-7.0 | KM | 0.1-7.0 |
| | AF189 | | AF190 | | AF191 |
| Emulsifier | 1.0-20 | Emulsifier | 1.0-20 | Emulsifier | 1.0-20 |
| Wax | 1.0-30 | Wax | 1.0-30 | Wax | 1.0-30 |
| (h + pi) (1:10) | 0.05-2 | (h + pi) + (h + ch) | 0.05-2 | (h + pi) + (h + a) | 0.05-2 |
| KM | 0.1-7.0 | KM | 0.1-7.0 | KM | 0.1-7.0 |
| | AF192 | | AF193 | | AF194 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (h + pi) (1:10) | 0.05-2 | (h + pi) + (h + ch) | 0.05-2 | (h + pi) + (h + a) | 0.05-2 |
| KM | 0.1-7.0 | KM | 0.1-7.0 | KM | 0.1-7.0 |
| | AF195 | | AF196 | | AF197 |
| Emulsifier | 1.0-20 | Emulsifier | 1.0-20 | Emulsifier | 1.0-20 |
| Oil | 1.0-30 | Oil | 1.0-30 | Wax | 1.0-30 |
| (h + ch) (1:3) | 0.05-2 | (h + ch) + (h + a) | 0.05-2 | (h + ch) (1:3) | 0.05-2 |
| KM | 0.1-7.0 | KM | 0.1-7.0 | KM | 0.1-7.0 |
| | AF198 | | AF199 | | AF200 |
| Emulsifier | 1.0-20 | Emulsifier | 1.0-20 | Emulsifier | 1.0-20 |
| Oil | 1.0-30 | Ester | 1.0-30 | Ester | 1.0-30 |
| (h + ch) + (h + a) | 0.05-2 | (h + ch) (1:3) | 0.05-2 | (h + ch) + (h + a) | 0.05-2 |
| KM | 0.1-7.0 | KM | 0.1-7.0 | KM | 0.1-7.0 |
| | AF201 | | AF202 | | AF203 |
| Emulsifier | 1.0-20 | Emulsifier | 1.0-20 | Emulsifier | 1.0-20 |
| Oil | 1.0-30 | Wax | 1.0-30 | Ester | 1.0-30 |
| (h + a) (1:5) | 0.05-2 | (h + a) (1:5) | 0.05-2 | (h + a) (1:5) | 0.05-2 |
| KM | 0.1-7.0 | KM | 0.1-7.0 | KM | 0.1-7.0 |
| | AF204 | | AF205 | | AF206 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF207 | | AF208 | | AF209 |
| Emulsifier | 1.0-20 | Emulsifier | 1.0-20 | Emulsifier | 1.0-20 |
| Ester | 1.0-30 | Ester | 1.0-30 | Ester | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF210 | | AF211 | | AF212 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF213 | | AF214 | | AF215 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (c + p) + (p + a) | 0.05-2 | (c + p) + (s2) + h) | 0.05-2 | (c + p) + (e + a) | 0.05-2 |
| KM | 0.1-7.0 | KM | 0.1-7.0 | KM | 0.1-7.0 |
| | AF216 | | AF217 | | AF218 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF219 | | AF220 | | AF221 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF222 | | AF223 | | AF224 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (u + a) (1:3) | 0.05-2 | (u + a) + (p + pi) | 0.05-2 | (u + a) + (p + a) | 0.05-2 |
| KM | 0.1-7.0 | KM | 0.1-7.0 | KM | 0.1-7.0 |
| | AF225 | | AF226 | | AF227 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (u + a) + ( s2) + h) | 0.05-2 | (u + a) + (e + a) | 0.05-2 | (u + a) + (e + c) | 0.05-2 |
| KM | 0.1-7.0 | KM | 0.1-7.0 | KM | 0.1-7.0 |
| | AF228 | | AF229 | | AF230 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (u + a) + (h + b) | 0.05-2 | (u + a) + (h + c) | 0.05-2 | (u + a) + (h + pi) | 0.05-2 |
| KM | 0.1-7.0 | KM | 0.1-7.0 | KM | 0.1-7.0 |
| | AF231 | | AF232 | | AF233 |
| Emulsifier | 1.0-20 | Emulsifier | 1.0-20 | Emulsifier | 1.0-20 |
| Oil + Wax | 1.0-30 | Oil + Wax | 1.0-30 | KationT | 1.0-30 |
| (u + a) + (h + ch) | 0.05-2 | (u + a) + (h + a) | 0.05-2 | (u + a) (1:3) | 0.05-2 |
| KM | 0.1-7.0 | KM | 0.1-7.0 | KM | 0.1-7.0 |
| | AF234 | | AF235 | | AF236 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (p + pi) (1:1) | 0.05-2 | (p + pi) + (p + a) | 0.05-2 | (p + pi) + (s2) + h) | 0.05-2 |
| KM | 0.1-7.0 | KM | 0.1-7.0 | KM | 0.1-7.0 |
| | AF237 | | AF238 | | AF239 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (p + pi) + (e + a) | 0.05-2 | (p + pi) + (e + c) | 0.05-2 | (p + pi) + (h + b) | 0.05-2 |
| KM | 0.1-7.0 | KM | 0.1-7.0 | KM | 0.1-7.0 |
| | AF240 | | AF241 | | AF242 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (p + pi) + (h + c) | 0.05-2 | (p + pi) + (h + pi) | 0.05-2 | (p + pi) + (h + ch) | 0.05-2 |
| KM | 0.1-7.0 | KM | 0.1-7.0 | KM | 0.1-7.0 |
| | AF243 | | AF244 | | AF245 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (p + pi) + (h + a) | 0.05-2 | (s2) + h) (2:1) | 0.05-2 | (s2) + h) + (e + a) | 0.05-2 |
| KM | 0.1-7.0 | KM | 0.1-7.0 | KM | 0.1-7.0 |
| | AF246 | | AF247 | | AF248 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (s2) + h) + (e + c) | 0.05-2 | (s2) + h) + (h + b) | 0.05-2 | (p + pi) + (e + c) | 0.05-2 |
| KM | 0.1-7.0 | KM | 0.1-7.0 | KM | 0.1-7.0 |
| | AF249 | | AF250 | | AF251 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (p + pi) + (h + b) | 0.05-2 | (s2) + h) + (h + c) | 0.05-2 | (s2) + h) + (h + pi) | 0.05-2 |
| KM | 0.1-7.0 | KM | 0.1-7.0 | KM | 0.1-7.0 |
| | AF252 | | AF253 | | AF254 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (s2) + h) + (h + ch) | 0.05-2 | (s2) + h) + (h + a) | 0.05-2 | (e + a) (1:2) | 0.05-2 |
| KM | 0.1-7.0 | KM | 0.1-7.0 | KM | 0.1-7.0 |
| | AF255 | | AF256 | | AF257 |
| Oil + Wax | 1.0-30 | Oil + Wax | 1.0-30 | Oil + Wax | 1.0-30 |
| (e + a) + (e + c) | 0.05-2 | (e + a) + (h + b) | 0.05-2 | (e + a) + (h + c) | 0.05-2 |
| KM | 0.1-7.0 | KM | 0.1-7.0 | KM | 0.1-7.0 |
| | AF258 | | AF259 | | AF260 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (e + a) + (h + pi) | 0.05-2 | (e + a) + (h + ch) | 0.05-2 | (e + a) + (h + a) | 0.05-2 |
| KM | 0.1-7.0 | KM | 0.1-7.0 | KM | 0.1-7.0 |
| | AF261 | | AF262 | | AF263 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF264 | | AF265 | | AF266 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF267 | | AF268 | | AF269 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF270 | | AF271 | | AF272 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF273 | | AF274 | | AF275 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (h + c) (1:5) | 0.05-2 | (h + c) + (h + pi) | 0.05-2 | (e + c) + (h + ch) | 0.05-2 |
| KM | 0.1-7.0 | KM | 0.1-7.0 | KM | 0.1-7.0 |
| | AF276 | | AF277 | | AF278 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (h + c) + (h + a) | 0.05-2 | (h + pi) (1:10) | 0.05-2 | (h + pi) + (h + ch) | 0.05-2 |
| KM | 0.1-7.0 | KM | 0.1-7.0 | KM | 0.1-7.0 |
| | AF279 | | AF280 | | AF281 |
| Emulsifier | 1.0-20 | Emulsifier | 1.0-20 | Emulsifier | 1.0-20 |
| Oil + Wax | 1.0-30 | Oil + Wax | 1.0-30 | Oil + Wax | 1.0-30 |
| (h + pi) + (h + a) | 0.05-2 | (h + ch) (1:3) | 0.05-2 | (h + ch) + (h + a) | 0.05-2 |
| KM | 0.1-7.0 | KM | 0.1-7.0 | KM | 0.1-7.0 |
| | AF282 | | AF283 | | |
| Emulsifier | 1.0-20 | Emulsifier | 1.0-20 | | |
| Oil + Wax | 1.0-30 | Oil + Wax | 1.0-30 | | |
| (h + ch) + (h + a) | 0.05-2 | (h + a) (1:5) | 0.05-2 | | |
| KM | 0.1-7.0 | KM | 0.1-7.0 | | |
| | AF284 | | AF285 | | AF286 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF287 | | AF288 | | AF289 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (c + p) + (p + a) | 0.05-2 | (c + p) + (s2) + h) | 0.05-2 | (c + p) + (e + a) | 0.05-2 |
| KM | 0.1-7.0 | KM | 0.1-7.0 | KM | 0.1-7.0 |
| | AF290 | | AF291 | | AF292 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF293 | | AF294 | | AF295 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF296 | | AF297 | | AF298 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (u + a) (1:3) | 0.05-2 | (u + a) + (p + pi) | 0.05-2 | (u + a) + (p + a) | 0.05-2 |
| KM | 0.1-7.0 | KM | 0.1-7.0 | KM | 0.1-7.0 |
| | AF299 | | AF300 | | AF301 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (u + a) + ( s2) + h) | 0.05-2 | (u + a) + (e + a) | 0.05-2 | (u + a) + (e + c) | 0.05-2 |
| KM | 0.1-7.0 | KM | 0.1-7.0 | KM | 0.1-7.0 |
| | AF302 | | AF303 | | AF304 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (u + a) + (h + b) | 0.05-2 | (u + a) + (h + c) | 0.05-2 | (u + a) + (h + pi) | 0.05-2 |
| KM | 0.1-7.0 | KM | 0.1-7.0 | KM | 0.1-7.0 |
| | AF305 | | AF306 | | AF307 |
| Emulsifier | 1.0-20 | Emulsifier | 1.0-20 | Emulsifier | 1.0-20 |
| Oil + Ester | 1.0-30 | Oil + Ester | 1.0-30 | KationT | 1.0-30 |
| (u + a) + (h + ch) | 0.05-2 | (u + a) + (h + a) | 0.05-2 | (u + a) (1:3) | 0.05-2 |
| KM | 0.1-7.0 | KM | 0.1-7.0 | KM | 0.1-7.0 |
| | AF308 | | AF309 | | AF310 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (p + pi) (1:1) | 0.05-2 | (p + pi) + (p + a) | 0.05-2 | (p + pi) + (s2) + h) | 0.05-2 |
| KM | 0.1-7.0 | KM | 0.1-7.0 | KM | 0.1-7.0 |
| | AF311 | | AF312 | | AF313 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (p + pi) + (e + a) | 0.05-2 | (p + pi) + (e + c) | 0.05-2 | (p + pi) + (h + b) | 0.05-2 |
| KM | 0.1-7.0 | KM | 0.1-7.0 | KM | 0.1-7.0 |
| | AF314 | | AF315 | | AF316 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (p + pi) + (h + c) | 0.05-2 | (p + pi) + (h + pi) | 0.05-2 | (p + pi) + (h + ch) | 0.05-2 |
| KM | 0.1-7.0 | KM | 0.1-7.0 | KM | 0.1-7.0 |
| | AF317 | | AF318 | | AF319 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (p + pi) + (h + a) | 0.05-2 | (s2) + h) (2:1) | 0.05-2 | (s2) + h) + (e + a) | 0.05-2 |
| KM | 0.1-7.0 | KM | 0.1-7.0 | KM | 0.1-7.0 |
| | AF320 | | AF321 | | AF322 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (s2) + h) + (e + c) | 0.05-2 | (s2) + h) + (h + b) | 0.05-2 | (p + pi) + (e + c) | 0.05-2 |
| KM | 0.1-7.0 | KM | 0.1-7.0 | KM | 0.1-7.0 |
| | AF323 | | AF324 | | AF325 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (p + pi) + (h + b) | 0.05-2 | (s2) + h) + (h + c) | 0.05-2 | (s2) + h) + (h + pi) | 0.05-2 |
| KM | 0.1-7.0 | KM | 0.1-7.0 | KM | 0.1-7.0 |
| | AF326 | | AF327 | | AF328 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (s2) + h) + (h + ch) | 0.05-2 | (s2) + h) + (h + a) | 0.05-2 | (e + a) (1:2) | 0.05-2 |
| KM | 0.1-7.0 | KM | 0.1-7.0 | KM | 0.1-7.0 |
| | AF329 | | AF330 | | AF331 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (e + a) + (e + c) | 0.05-2 | (e + a) + (h + b) | 0.05-2 | (e + a) + (h + c) | 0.05-2 |
| KM | 0.1-7.0 | KM | 0.1-7.0 | KM | 0.1-7.0 |
| | AF332 | | AF333 | | AF334 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (e + a) + (h + pi) | 0.05-2 | (e + a) + (h + ch) | 0.05-2 | (e + a) + (h + a) | 0.05-2 |
| KM | 0.1-7.0 | KM | 0.1-7.0 | KM | 0.1-7.0 |
| | AF335 | | AF336 | | AF337 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF338 | | AF339 | | AF340 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF341 | | AF342 | | AF343 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF344 | | AF345 | | AF346 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF347 | | AF348 | | AF349 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (h + c) (1:5) | 0.05-2 | (h + c) + (h + pi) | 0.05-2 | (e + c) + (h + ch) | 0.05-2 |
| KM | 0.1-7.0 | KM | 0.1-7.0 | KM | 0.1-7.0 |
| | AF350 | | AF351 | | AF352 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (h + c) + (h + a) | 0.05-2 | (h + pi) (1:10) | 0.05-2 | (h + pi) + (h + ch) | 0.05-2 |
| KM | 0.1-7.0 | KM | 0.1-7.0 | KM | 0.1-7.0 |
| | AF353 | | AF354 | | AF355 |
| Emulsifier | 1.0-20 | Emulsifier | 1.0-20 | Emulsifier | 1.0-20 |
| Oil + Ester | 1.0-30 | Oil + Ester | 1.0-30 | Oil + Ester | 1.0-30 |
| (h + pi) + (h + a) | 0.05-2 | (h + ch) (1:3) | 0.05-2 | (h + ch) + (h + a) | 0.05-2 |
| KM | 0.1-7.0 | KM | 0.1-7.0 | KM | 0.1-7.0 |
| | AF356 | | AF357 | | |
| Emulsifier | 1.0-20 | Emulsifier | 1.0-20 | | |
| Oil + Ester | 1.0-30 | Oil + Ester | 1.0-30 | | |
| (h + ch) + (h + a) | 0.05-2 | (h + a) (1:5) | 0.05-2 | | |
| KM | 0.1-7.0 | KM | 0.1-7.0 | | |
| | AF358 | | AF359 | | AF360 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF361 | | AF362 | | AF363 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (c + p) + (p + a) | 0.05-2 | (c + p) + (s2) + h) | 0.05-2 | (c + p) + (e + a) | 0.05-2 |
| KM | 0.1-7.0 | KM | 0.1-7.0 | KM | 0.1-7.0 |
| | AF364 | | AF365 | | AF366 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF367 | | AF368 | | AF369 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF370 | | AF371 | | AF372 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (u + a) (1:3) | 0.05-2 | (u + a) + (p + pi) | 0.05-2 | (u + a) + (p + a) | 0.05-2 |
| KM | 0.1-7.0 | KM | 0.1-7.0 | KM | 0.1-7.0 |
| | AF373 | | AF374 | | AF375 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (u + a) + ( s2) + h) | 0.05-2 | (u + a) + (e + a) | 0.05-2 | (u + a) + (e + c) | 0.05-2 |
| KM | 0.1-7.0 | KM | 0.1-7.0 | KM | 0.1-7.0 |
| | AF376 | | AF377 | | AF378 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (u + a) + (h + b) | 0.05-2 | (u + a) + (h + c) | 0.05-2 | (u + a) + (h + pi) | 0.05-2 |
| KM | 0.1-7.0 | KM | 0.1-7.0 | KM | 0.1-7.0 |
| | AF379 | | AF380 | | AF381 |
| Emulsifier | 1.0-20 | Emulsifier | 1.0-20 | Emulsifier | 1.0-20 |
| Ester + Wax | 1.0-30 | Ester + Wax | 1.0-30 | KationT | 1.0-30 |
| (u + a) + (h + ch) | 0.05-2 | (u + a) + (h + a) | 0.05-2 | (u + a) (1:3) | 0.05-2 |
| KM | 0.1-7.0 | KM | 0.1-7.0 | KM | 0.1-7.0 |
| | AF382 | | AF383 | | AF384 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (p + pi) (1:1) | 0.05-2 | (p + pi) + (p + a) | 0.05-2 | (p + pi) + (s2) + h) | 0.05-2 |
| KM | 0.1-7.0 | KM | 0.1-7.0 | KM | 0.1-7.0 |
| | AF385 | | AF386 | | AF387 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (p + pi) + (e + a) | 0.05-2 | (p + pi) + (e + c) | 0.05-2 | (p + pi) + (h + b) | 0.05-2 |
| KM | 0.1-7.0 | KM | 0.1-7.0 | KM | 0.1-7.0 |
| | AF388 | | AF389 | | AF390 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (p + pi) + (h + c) | 0.05-2 | (p + pi) + (h + pi) | 0.05-2 | (p + pi) + (h + ch) | 0.05-2 |
| KM | 0.1-7.0 | KM | 0.1-7.0 | KM | 0.1-7.0 |
| | AF391 | | AF392 | | AF393 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (p + pi) + (h + a) | 0.05-2 | (s2) + h) (2:1) | 0.05-2 | (s2) + h) + (e + a) | 0.05-2 |
| KM | 0.1-7.0 | KM | 0.1-7.0 | KM | 0.1-7.0 |
| | AF394 | | AF395 | | AF396 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (s2) + h) + (e + c) | 0.05-2 | (s2) + h) + (h + b) | 0.05-2 | (p + pi) + (e + c) | 0.05-2 |
| KM | 0.1-7.0 | KM | 0.1-7.0 | KM | 0.1-7.0 |
| | AF397 | | AF398 | | AF399 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (p + pi) + (h + b) | 0.05-2 | (s2) + h) + (h + c) | 0.05-2 | (s2) + h) + (h + pi) | 0.05-2 |
| KM | 0.1-7.0 | KM | 0.1-7.0 | KM | 0.1-7.0 |
| | AF400 | | AF401 | | AF402 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (s2) + h) + (h + ch) | 0.05-2 | (s2) + h) + (h + a) | 0.05-2 | (e + a) (1:2) | 0.05-2 |
| KM | 0.1-7.0 | KM | 0.1-7.0 | KM | 0.1-7.0 |
| | AF403 | | AF404 | | AF405 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (e + a) + (e + c) | 0.05-2 | (e + a) + (h + b) | 0.05-2 | (e + a) + (h + c) | 0.05-2 |
| KM | 0.1-7.0 | KM | 0.1-7.0 | KM | 0.1-7.0 |
| | AF406 | | AF407 | | AF408 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (e + a) + (h + pi) | 0.05-2 | (e + a) + (h + ch) | 0.05-2 | (e + a) + (h + a) | 0.05-2 |
| KM | 0.1-7.0 | KM | 0.1-7.0 | KM | 0.1-7.0 |
| | AF409 | | AF410 | | AF411 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF412 | | AF413 | | AF414 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF415 | | AF416 | | AF417 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF418 | | AF419 | | AF420 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF421 | | AF422 | | AF423 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (h + c) (1:5) | 0.05-2 | (h + c) + (h + pi) | 0.05-2 | (e + c) + (h + ch) | 0.05-2 |
| KM | 0.1-7.0 | KM | 0.1-7.0 | KM | 0.1-7.0 |
| | AF424 | | AF425 | | AF426 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (h + c) + (h + a) | 0.05-2 | (h + pi) (1:10) | 0.05-2 | (h + pi) + (h + ch) | 0.05-2 |
| KM | 0.1-7.0 | KM | 0.1-7.0 | KM | 0.1-7.0 |
| | AF427 | | AF428 | | AF429 |
| Emulsifier | 1.0-20 | Emulsifier | 1.0-20 | Emulsifier | 1.0-20 |
| Ester + Wax | 1.0-30 | Ester + Wax | 1.0-30 | Ester + Wax | 1.0-30 |
| (h + pi) + (h + a) | 0.05-2 | (h + ch) (1:3) | 0.05-2 | (h + ch) + (h + a) | 0.05-2 |
| KM | 0.1-7.0 | KM | 0.1-7.0 | KM | 0.1-7.0 |
| | AF430 | | AF431 | | |
| Emulsifier | 1.0-20 | Emulsifier | 1.0-20 | | |
| Ester + Wax | 1.0-30 | Ester + Wax | 1.0-30 | | |
| (h + ch) + (h + a) | 0.05-2 | (h + a) (1:5) | 0.05-2 | | |
| KM | 0.1-7.0 | KM | 0.1-7.0 | | |
| | AF432 | | AF433 | | AF434 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (c + p) (1:2) | 0.05-2 | (c + p) + (u + a) | 0.05-2 | (c + p) + (p + pi) | 0.05-2 |
| KM | 0.1-7.0 | KM | 0.1-7.0 | KM | 0.1-7.0 |
| | AF435 | | AF436 | | AF437 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (c + p) + (p + a) | 0.05-2 | (c + p) + (s2) + h) | 0.05-2 | (c + p) + (e + a) | 0.05-2 |
| KM | 0.1-7.0 | KM | 0.1-7.0 | KM | 0.1-7.0 |
| | AF438 | | AF439 | | AF440 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (c + p) + (e + c) | 0.05-2 | (c + p) + (h + b) | 0.05-2 | (c + p) + (h + c) | 0.05-2 |
| KM | 0.1-7.0 | KM | 0.1-7.0 | KM | 0.1-7.0 |
| | AF441 | | AF442 | | AF443 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (c + p) + (h + pi) | 0.05-2 | (c + p) + (h + ch) | 0.05-2 | (c + p) + (h + a) | 0.05-2 |
| KM | 0.1-7.0 | KM | 0.1-7.0 | KM | 0.1-7.0 |
| | AF444 | | AF445 | | AF446 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (u + a) (1:3) | 0.05-2 | (u + a) + (p + pi) | 0.05-2 | (u + a) + (p + a) | 0.05-2 |
| KM | 0.1-7.0 | KM | 0.1-7.0 | KM | 0.1-7.0 |
| | AF447 | | AF448 | | AF449 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (u + a) + ( s2) + h) | 0.05-2 | (u + a) + (e + a) | 0.05-2 | (u + a) + (e + c) | 0.05-2 |
| KM | 0.1-7.0 | KM | 0.1-7.0 | KM | 0.1-7.0 |
| | AF450 | | AF451 | | AF452 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (u + a) + (h + b) | 0.05-2 | (u + a) + (h + c) | 0.05-2 | (u + a) + (h + pi) | 0.05-2 |
| KM | 0.1-7.0 | KM | 0.1-7.0 | KM | 0.1-7.0 |
| | AF453 | | AF454 | | AF455 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | KationT | 1.0-30 |
| (u + a) + (h + ch) | 0.05-2 | (u + a) + (h + a) | 0.05-2 | (u + a) (1:3) | 0.05-2 |
| KM | 0.1-7.0 | KM | 0.1-7.0 | KM | 0.1-7.0 |
| | AF456 | | AF457 | | AF458 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (p + pi) (1:1) | 0.05-2 | (p + pi) + (p + a) | 0.05-2 | (p + pi) + (s2) + h) | 0.05-2 |
| KM | 0.1-7.0 | KM | 0.1-7.0 | KM | 0.1-7.0 |
| | AF459 | | AF460 | | AF461 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (p + pi) + (e + a) | 0.05-2 | (p + pi) + (e + c) | 0.05-2 | (p + pi) + (h + b) | 0.05-2 |
| KM | 0.1-7.0 | KM | 0.1-7.0 | KM | 0.1-7.0 |
| | AF462 | | AF463 | | AF464 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (p + pi) + (h + c) | 0.05-2 | (p + pi) + (h + pi) | 0.05-2 | (p + pi) + (h + ch) | 0.05-2 |
| KM | 0.1-7.0 | KM | 0.1-7.0 | KM | 0.1-7.0 |
| | AF465 | | AF466 | | AF467 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (p + pi) + (h + a) | 0.05-2 | (s2) + h) (2:1) | 0.05-2 | (s2) + h) + (e + a) | 0.05-2 |
| KM | 0.1-7.0 | KM | 0.1-7.0 | KM | 0.1-7.0 |
| | AF468 | | AF469 | | AF470 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (s2) + h) + (e + c) | 0.05-2 | (s2) + h) + (h + b) | 0.05-2 | (p + pi) + (e + c) | 0.05-2 |
| KM | 0.1-7.0 | KM | 0.1-7.0 | KM | 0.1-7.0 |
| | AF471 | | AF472 | | AF473 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (p + pi) + (h + b) | 0.05-2 | (s2) + h) + (h + c) | 0.05-2 | (s2) + h) + (h + pi) | 0.05-2 |
| KM | 0.1-7.0 | KM | 0.1-7.0 | KM | 0.1-7.0 |
| | AF474 | | AF475 | | AF476 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (s2) + h) + (h + ch) | 0.05-2 | (s2) + h) + (h + a) | 0.05-2 | (e + a) (1:2) | 0.05-2 |
| KM | 0.1-7.0 | KM | 0.1-7.0 | KM | 0.1-7.0 |
| | AF477 | | AF478 | | AF479 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (e + a) + (e + c) | 0.05-2 | (e + a) + (h + b) | 0.05-2 | (e + a) + (h + c) | 0.05-2 |
| KM | 0.1-7.0 | KM | 0.1-7.0 | KM | 0.1-7.0 |
| | AF480 | | AF481 | | AF482 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (e + a) + (h + pi) | 0.05-2 | (e + a) + (h + ch) | 0.05-2 | (e + a) + (h + a) | 0.05-2 |
| KM | 0.1-7.0 | KM | 0.1-7.0 | KM | 0.1-7.0 |
| | AF483 | | AF484 | | AF485 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (e + c) (1:2) | 0.05-2 | (e + c) + (h + b) | 0.05-2 | (e + c) + (h + c) | 0.05-2 |
| KM | 0.1-7.0 | KM | 0.1-7.0 | KM | 0.1-7.0 |
| | AF486 | | AF487 | | AF488 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (e + c) + (h + pi) | 0.05-2 | (e + c) + (h + ch) | 0.05-2 | (e + c) + (h + a) | 0.05-2 |
| KM | 0.1-7.0 | KM | 0.1-7.0 | KM | 0.1-7.0 |
| | AF489 | | AF490 | | AF491 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (h + b) (1:10) | 0.05-2 | (h + b) + (h + c) | 0.05-2 | (e + c) + (h + pi) | 0.05-2 |
| KM | 0.1-7.0 | KM | 0.1-7.0 | KM | 0.1-7.0 |
| | AF492 | | AF493 | | AF494 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (h + b) + (h + pi) | 0.05-2 | (h + b) + (h + ch) | 0.05-2 | (e + c) + (h + a) | 0.05-2 |
| KM | 0.1-7.0 | KM | 0.1-7.0 | KM | 0.1-7.0 |
| | AF495 | | AF496 | | AF497 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (h + c) (1:5) | 0.05-2 | (h + c) + (h + pi) | 0.05-2 | (e + c) + (h + ch) | 0.05-2 |
| KM | 0.1-7.0 | KM | 0.1-7.0 | KM | 0.1-7.0 |
| | AF498 | | AF499 | | AF500 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (h + c) + (h + a) | 0.05-2 | (h + pi) (1:10) | 0.05-2 | (h + pi) + (h + ch) | 0.05-2 |
| KM | 0.1-7.0 | KM | 0.1-7.0 | KM | 0.1-7.0 |
| | AF501 | | AF502 | | AF503 |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | Ester + Wax + Oil | 1.0-30 |
| (h + pi) + (h + a) | 0.05-2 | (h + ch) (1:3) | 0.05-2 | (h + ch) + (h + a) | 0.05-2 |
| KM | 0.1-7.0 | KM | 0.1-7.0 | KM | 0.1-7.0 |
| | AF504 | | AF505 | | |
| Emulsifier | 1.0-20 | Emulsifier | 1.0-20 | Emulsifier | 1.0-20 |
| Ester + Wax + Oil | 1.0-30 | Ester + Wax + Oil | 1.0-30 | | |
| (h + ch) + (h + a) | 0.05-2 | (h + a) (1:5) | 0.05-2 | | |
| KM | 0.1-7.0 | KM | 0.1-7.0 | | |

²⁾or 2): Sulfite is selected from the group consisting of sodium sulfite, ammonium bisulfite, ammonium sulfite, potassium sulfite, potassium hydrogen sulfite, sodium bisulfite, sodium metabisulfite, and potassium metabisulfite, and mixtures thereof

The aforementioned oils, waxes, and esters are preferably used as the oil, wax, and ester in the embodiments AF1 to AF505. The cosmetic agents AF1 to AF505 have a favorable cleaning effect and excellent preservation. The synergistic effect of the preservatives used makes it possible to reduce the amount used without negatively affecting the preservative effect. The cosmetic compositions according to the present invention are therefore low-irritant and low-sensitization.

In addition to the components a) to d) that are compulsory according to the present invention, the cosmetic agents according to the present invention may in principle include any and all other components known to a person skilled in the art for such cosmetic compositions. Examples of further active ingredients, auxiliary substances, and additives include:
thickening agents such as gelatins or plant gums, for example agar-agar, guar gum, alginates, xanthan gum, gum arabic, karaya gum, carob seed meal, linseed gums, dextrans, cellulose derivatives, for example methyl cellulose, hydroxyalkyl cellulose, and carboxymethyl cellulose, starch fractions and derivatives such as amylose, amylopectin, and dextrins, fully synthetic hydrocolloids such as, for example, polyvinyl alcohol;
texturizing agents such as maleic acid and lactic acid;
solvents and solubilizers such as ethanol, isopropanol, ethylene glycol, propylene glycol, glycerol, and diethylene glycol;
active ingredients to improve the fiber structure, in particular mono-, di- and oligosaccharides such as, for example, glucose, galactose, fructose, fruit sugar, and lactose;
dyes to color the agent;
substances for adjusting the pH value, such as α- and β-hydroxycarboxylic acids;
active ingredients such as allantoin and bisabolol;
complexing agents such as EDTA, NTA, β-alanine diacetic acid, and phosphonic acids;
ceramides. Ceramides are understood to be N-acyl sphingosine (fatty acid amides of sphingosine) or synthetic analogs of such lipids (known as pseudoceramides),
opacifiers such as latex, styrene/PVP and styrene/acrylamide copolymers;
pearling agents such as ethylene glycol mono- and distearate, as well as PEG-3 distearate;
pigments;
propellants such as propane-butane mixtures, N₂O, dimethyl ether, CO₂, and air;
viscosity adjusters such as salts (NaCl);
anionic, cationic, and amphoteric surfactants;
cationic, nonionic, and amphoteric polymers;
vitamins, in particular from the group consisting of A, B, C, E, F, and H;
UV filters, in particular, benzophenones, p-aminobenzoic acid esters, diphenyl acrylic acid esters, cinnamic acid esters, salicylic acid esters, benzimidazoles, and o-aminobenzoic acid esters;
protein hydrolysates and cationized protein hydrolysates;
humectants or penetration enhancers and/or swelling agents, in particular, urea and urea derivatives, guanidine and derivatives thereof, arginine and derivatives thereof, water glass, imidazole and derivatives thereof, histidine and derivatives thereof, benzyl alcohol, glycol ethers, propylene glycol ethers, for example propylene glycol monoethyl ether, carbonates, hydrogen carbonates, 1,2-diols, and 1,3-diols;
plant extracts, e.g., from green tea, white tea, oak bark, stinging nettle, witch hazel, hops, chamomile, burdock, horsetail, whitethorn, lime blossom, lychee, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, ginseng, ginger root, Echinacea purpurea, Olea europaea, Foeniculum vulgaris and Apium graveolens;
silicone oils, in particular, polyalkyl siloxanes, polyaryl siloxanes, and polyalkyl aryl siloxanes, which may optionally be functionalized with organic groups and/or ethoxy groups and/or propoxy groups.

The aforementioned further ingredients may be included—based on the total weight of the cosmetic agent—in a total amount of 0.001 to 50 wt %, preferably 0.01 to 40 wt %, preferably 0.1 to 30 wt %, in particular, 0.5 to 20 wt %.

A second subject matter of the present invention is the use of the cosmetic agents according to the present invention to clean and care for skin and hair.

What has been said about the cosmetic agents according to the present invention applies, mutatis mutandis, to further preferred embodiments of the use according to the present invention, in particular, regarding the cosmetic agents used.

The following examples illustrate the present invention, but do so in a non-limiting manner.

### Examples

The following cleansing emulsions were produced:

Cleansing milk (indicated in wt %)

| Raw material | 1.1 | 1.2 | 1.3 | 1.4 | 1.5 | 1.6 |
|---|---|---|---|---|---|---|
| Helianthus annuus seed oil | 5.00 | 5.00 | 5.00 | 5.00 | 5.00 | 5.00 |
| Isopropyl palmitate | 4.98 | 4.98 | 4.98 | 4.98 | 4.98 | 4.98 |
| Rosa damascena flower water | 4.97 | 4.97 | 4.97 | 4.97 | 4.97 | 4.97 |
| Ethyl lauroyl arginate * HCl | — | — | — | — | — | 0.40 |
| Piroctone olamine | — | — | — | — | — | 1.00 |
| Sodium sulfite | — | 0.20 | — | 0.20 | — | 0.20 |
| Hexetidine | 0.10 | 0.10 | — | — | — | 0.10 |
| Formic acid | — | — | 0.50 | — | — | 0.50 |
| Phenoxyisopropanol | — | — | 1.00 | — | 0.80 | 1.00 |
| Undecylenic acid | 0.20 | — | — | — | 0.20 | 0.20 |
| Chloroxylenol | — | — | — | 0.50 | — | 0.50 |
| Benzyl alcohol | — | — | — | — | — | 1.00 |
| Chlorphenesin | — | — | — | — | — | 0.30 |
| Benzoic acid | — | — | — | — | — | 0.20 |
| Phenoxyethanol | — | 0.50 | 0.40 | — | — | 0.50 |
| Methylparaben | 0.20 | — | — | — | 0.20 | 0.20 |
| Ethylparaben | 0.20 | — | — | — | 0.10 | 0.10 |
| Propionic acid | — | — | — | — | — | 2.00 |
| Sorbic acid | — | — | — | — | — | 0.60 |
| Polyaminopropyl biguanide | 0.30 | — | 0.30 | — | 0.30 | 0.30 |
| Climbazole | — | 0.50 | — | — | — | 0.50 |
| Chlorhexidine | — | — | — | — | 0.30 | 0.30 |
| Glutaraldehyde | — | — | 0.10 | — | — | 0.10 |
| Salicylic acid | 0.20 | — | — | — | — | 0.50 |
| Benzalkonium chloride | — | 0.10 | — | — | — | 0.10 |
| KM¹⁾ | — | — | — | 1.5 | — | — |
| Cetearyl alcohol | 2.50 | 2.50 | 2.50 | 2.50 | 2.50 | 2.50 |
| Cocoglycerides | 2.00 | 2.00 | 2.00 | 2.00 | 2.00 | 2.00 |
| Laurus nobilis leaf extract | 1.99 | 1.99 | 1.99 | 1.99 | 1.99 | 1.99 |
| Hydrogenated vegetable oil | 1.00 | 1.00 | 1.00 | 1.00 | 1.00 | 1.00 |
| Xanthan gum | 1.00 | 1.00 | 1.00 | 1.00 | 1.00 | 1.00 |
| Lauryl glucoside | 0.95 | 0.95 | 0.95 | 0.95 | 0.95 | 0.95 |
| Polyglyceryl-2 | 0.95 | 0.95 | 0.95 | 0.95 | 0.95 | 0.95 |
| dipolyhydroxystearate | | | | | | |
| Panthenol | 0.50 | 0.50 | 0.50 | 0.50 | 0.50 | 0.50 |
| Citric acid | 0.21 | 0.21 | 0.21 | 0.21 | 0.21 | 0.21 |
| Sodium stearoyl glutamate | 0.10 | 0.10 | 0.10 | 0.10 | 0.10 | 0.10 |
| Tocopheryl acetate | 0.09 | 0.09 | 0.09 | 0.09 | 0.09 | 0.09 |
| Citric acid | 0.02 | 0.02 | 0.02 | 0.02 | 0.02 | 0.02 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Cleansing milk (indicated in wt %)

| Raw material | 2.1 | 2.2 | 2.3 | 2.4 | 2.5 | 2.6 |
|---|---|---|---|---|---|---|
| Paraffinum liquidum | 20.00 | 20.00 | 20.00 | 20.00 | 20.00 | 20.00 |
| Glycerol | 1.99 | 1.99 | 1.99 | 1.99 | 1.99 | 1.99 |
| Ceteareth-12 | 1.39 | 1.39 | 1.39 | 1.39 | 1.39 | 1.39 |
| Triceteareth-4 phosphate | 1.27 | 1.27 | 1.27 | 1.27 | 1.27 | 1.27 |
| Cetearyl alcohol | 0.75 | 0.75 | 0.75 | 0.75 | 0.75 | 0.75 |
| Ethyl lauroyl arginate * HCl | 0.13 | — | — | — | 0.40 | 0.40 |
| Piroctone olamine | — | — | — | — | — | 1.00 |
| Sodium sulfite | — | — | — | — | — | 0.20 |
| Hexetidine | — | — | — | 0.10 | 0.10 | 0.10 |
| Formic acid | — | — | 0.50 | — | — | 0.50 |
| Phenoxyisopropanol | — | 1.00 | — | — | — | 1.00 |
| Undecylenic acid | — | — | 0.20 | — | — | 0.20 |
| Chloroxylenol | 0.75 | 0.50 | — | — | — | 0.50 |
| Benzyl alcohol | — | — | — | 1.00 | — | 1.00 |
| Chlorphenesin | — | — | — | — | — | 0.30 |
| Benzoic acid | 0.30 | — | — | — | — | 0.20 |
| Phenoxyethanol | — | — | — | — | — | 0.50 |
| Methylparaben | — | — | — | — | — | 0.20 |
| Ethylparaben | — | — | 0.10 | — | — | 0.10 |
| Propionic acid | — | — | — | — | — | 2.00 |
| Sorbic acid | — | 0.60 | — | — | — | 0.60 |
| Polyaminopropyl biguanide | — | — | — | — | — | 0.30 |
| Climbazole | — | — | — | — | — | 0.50 |
| Chlorhexidine | — | — | — | — | — | 0.30 |
| Glutaraldehyde | — | — | — | — | — | 0.10 |
| Salicylic acid | — | 0.20 | 0.20 | — | — | 0.50 |
| Benzalkonium chloride | — | — | — | — | — | 0.10 |
| KM¹⁾ | — | — | — | 0.5 | 2.0 | — |
| Sorbitol | 0.70 | 0.70 | 0.70 | 0.70 | 0.70 | 0.70 |
| Panthenol | 0.50 | 0.50 | 0.50 | 0.50 | 0.50 | 0.50 |
| Carbomer | 0.30 | 0.30 | 0.30 | 0.30 | 0.30 | 0.30 |
| Fragrance | 0.14 | 0.14 | 0.14 | 0.14 | 0.14 | 0.14 |
| Argania spinosa kernel oil | 0.10 | 0.10 | 0.10 | 0.10 | 0.10 | 0.10 |
| Xanthan gum | 0.09 | 0.09 | 0.09 | 0.09 | 0.09 | 0.09 |
| Trisodium dicarboxymethyl | 0.04 | 0.04 | 0.04 | 0.04 | 0.04 | 0.04 |
| alaninate | | | | | | |
| Pantolactone | 0.01 | 0.01 | 0.01 | 0.01 | 0.01 | 0.01 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Cleansing milk (indicated in wt %)

| Raw material | 3.1 | 3.2 | 3.3 | 3.4 | 3.5 | 3.6 |
|---|---|---|---|---|---|---|
| Ethylhexyl palmitate | 6.00 | 6.00 | 6.00 | 6.00 | 6.00 | 6.00 |
| Hexanediol | 6.00 | 6.00 | 6.00 | 6.00 | 6.00 | 6.00 |
| Hexyl laurate | 3.00 | 3.00 | 3.00 | 3.00 | 3.00 | 3.00 |
| Myristyl myristate | 2.91 | 2.91 | 2.91 | 2.91 | 2.91 | 2.91 |
| Caprylic/Capric triglyceride | 2.00 | 2.00 | 2.00 | 2.00 | 2.00 | 2.00 |
| Sucrose stearate | 1.91 | 1.91 | 1.91 | 1.91 | 1.91 | 1.91 |
| CI 77891 | 0.60 | 0.60 | 0.60 | 0.60 | 0.60 | 0.60 |
| Panthenol | 0.50 | 0.50 | 0.50 | 0.50 | 0.50 | 0.50 |
| Cetearyl alcohol | 0.50 | 0.50 | 0.50 | 0.50 | 0.50 | 0.50 |
| Ethyl lauroyl arginate * HCl | 0.40 | — | 0.40 | — | — | 0.40 |
| Piroctone olamine | — | 1.00 | — | — | — | 1.00 |
| Sodium sulfite | — | — | — | — | — | 0.20 |
| Hexetidine | — | — | — | — | — | 0.10 |
| Formic acid | 0.50 | — | 0.50 | 0.50 | — | 0.50 |
| Phenoxyisopropanol | — | 1.00 | — | — | 1.00 | 1.00 |
| Undecylenic acid | — | — | — | 0.20 | — | 0.20 |
| Chloroxylenol | — | — | — | — | 0.50 | 0.50 |
| Benzyl alcohol | — | — | — | — | — | 1.00 |
| Chlorphenesin | — | — | — | — | — | 0.30 |
| Benzoic acid | — | 0.20 | 0.20 | — | — | 0.20 |
| Phenoxyethanol | 0.50 | — | 0.50 | — | — | 0.50 |
| Methylparaben | — | — | — | — | — | 0.20 |
| Ethylparaben | — | — | — | — | — | 0.10 |
| Propionic acid | — | — | — | — | — | 2.00 |
| Sorbic acid | — | — | 0.60 | — | — | 0.60 |
| Polyaminopropyl biguanide | — | — | — | — | — | 0.30 |
| Climbazole | — | 0.50 | — | — | — | 0.50 |
| Chlorhexidine | — | — | — | — | — | 0.30 |
| Glutaraldehyde | — | — | — | — | — | 0.10 |
| Salicylic acid | — | — | — | — | — | 0.50 |
| Benzalkonium chloride | — | 0.10 | — | — | — | 0.10 |
| KM¹⁾ | — | — | — | 0.2 | 1.5 | — |
| Glyceryl stearate | 0.50 | 0.50 | 0.50 | 0.50 | 0.50 | 0.50 |
| Cera alba | 0.50 | 0.50 | 0.50 | 0.50 | 0.50 | 0.50 |
| Fragrance | 0.34 | 0.34 | 0.34 | 0.34 | 0.34 | 0.34 |
| Tocopheryl acetate | 0.30 | 0.30 | 0.30 | 0.30 | 0.30 | 0.30 |
| Hydroxypropyl | 0.20 | 0.20 | 0.20 | 0.20 | 0.20 | 0.20 |
| methylcellulose | | | | | | |
| Carbomer | 0.17 | 0.17 | 0.17 | 0.17 | 0.17 | 0.17 |
| Bisabolol | 0.09 | 0.09 | 0.09 | 0.09 | 0.09 | 0.09 |
| Myristyl alcohol | 0.09 | 0.09 | 0.09 | 0.09 | 0.09 | 0.09 |
| Sodium hydroxide | 0.06 | 0.06 | 0.06 | 0.06 | 0.06 | 0.06 |
| Retinyl palmitate | 0.06 | 0.06 | 0.06 | 0.06 | 0.06 | 0.06 |
| Arachis hypogaea oil | 0.04 | 0.04 | 0.04 | 0.04 | 0.04 | 0.04 |
| Stearic acid | 0.03 | 0.03 | 0.03 | 0.03 | 0.03 | 0.03 |
| Tocopherol | 0.03 | 0.03 | 0.03 | 0.03 | 0.03 | 0.03 |
| Hydrogenated palm glycerides | 0.02 | 0.02 | 0.02 | 0.02 | 0.02 | 0.02 |
| citrate | | | | | | |
| Sucrose | 0.02 | 0.02 | 0.02 | 0.02 | 0.02 | 0.02 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Washing cream (All values represent wt %)

| Raw material | 4.1 | 4.2 | 4.3 | 4.4 | 4.5 | 4.6 |
|---|---|---|---|---|---|---|
| Coco-caprylate/Caprate | 5.00 | 5.00 | 5.00 | 5.00 | 5.00 | 5.00 |
| Dicaprylyl carbonate | 5.00 | 5.00 | 5.00 | 5.00 | 5.00 | 5.00 |
| Pentaerythrityl distearate | 1.00 | 1.00 | 1.00 | 1.00 | 1.00 | 1.00 |
| Propylene glycol | 0.84 | 0.84 | 0.84 | 0.84 | 0.84 | 0.84 |
| Sodium polyacrylate | 0.50 | 0.50 | 0.50 | 0.50 | 0.50 | 0.50 |
| Ethyl lauroyl arginate * HCl | 0.40 | — | — | 0.40 | — | 0.40 |
| Piroctone olamine | — | — | — | — | 0.50 | 1.00 |
| Sodium sulfite | — | — | — | — | — | 0.20 |
| Hexetidine | 0.10 | 0.10 | 0.10 | — | — | 0.10 |
| Formic acid | — | — | — | — | — | 0.50 |
| Phenoxyisopropanol | — | — | — | — | 1.00 | 1.00 |
| Undecylenic acid | — | — | — | 0.20 | — | 0.20 |
| Chloroxylenol | — | — | 0.50 | — | — | 0.50 |
| Benzyl alcohol | — | 1.00 | — | — | — | 1.00 |
| Chlorphenesin | — | — | — | — | — | 0.30 |
| Benzoic acid | — | — | — | — | — | 0.20 |
| Phenoxyethanol | — | 0.40 | 0.40 | — | — | 0.50 |
| Methylparaben | — | — | — | — | — | 0.20 |
| Ethylparaben | — | — | — | — | — | 0.10 |
| Propionic acid | — | — | — | — | — | 2.00 |
| Sorbic acid | 0.20 | — | — | — | — | 0.60 |
| Polyaminopropyl biguanide | — | — | 0.30 | — | — | 0.30 |
| Climbazole | — | 0.50 | — | — | — | 0.50 |
| Chlorhexidine | — | — | — | — | — | 0.30 |
| Glutaraldehyde | — | — | 0.10 | — | — | 0.10 |
| Salicylic acid | — | — | — | — | — | 0.50 |
| Benzalkonium chloride | — | 0.10 | — | — | — | 0.10 |
| KM¹⁾ | — | — | — | 0.1 | 1.0 | — |
| Fragrance | 0.32 | 0.32 | 0.32 | 0.32 | 0.32 | 0.32 |
| Tetrasodium EDTA | 0.10 | 0.10 | 0.10 | 0.10 | 0.10 | 0.10 |
| Butylene glycol | 0.09 | 0.09 | 0.09 | 0.09 | 0.09 | 0.09 |
| Pentylene glycol | 0.04 | 0.04 | 0.04 | 0.04 | 0.04 | 0.04 |
| Hydrolyzed soy protein | 0.03 | 0.03 | 0.03 | 0.03 | 0.03 | 0.03 |
| Prunus persica fruit extract | 0.02 | 0.02 | 0.02 | 0.02 | 0.02 | 0.02 |
| Panax ginseng root extract | 0.01 | 0.01 | 0.01 | 0.01 | 0.01 | 0.01 |
| Tocopherol | 0.00 | 0.00 | 0.00 | 0.00 | 0.00 | 0.00 |
| Sebacic acid | 0.00 | 0.00 | 0.00 | 0.00 | 0.00 | 0.00 |
| 10-Hydroxydecanoic acid | 0.00 | 0.00 | 0.00 | 0.00 | 0.00 | 0.00 |
| 1,10-Decanediol | 0.00 | 0.00 | 0.00 | 0.00 | 0.00 | 0.00 |
| 2,4-Dimethyl-3-cyclohexene | 0.00 | 0.00 | 0.00 | 0.00 | 0.00 | 0.00 |
| carboxaldehyde | | | | | | |
| Pinus sylvestris bark extract | 0.00 | 0.00 | 0.00 | 0.00 | 0.00 | 0.00 |
| Ribes nigrum leaf extract | 0.00 | 0.00 | 0.00 | 0.00 | 0.00 | 0.00 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Cleansing milk cold production (all values represent wt %)

| Raw material | 5.1 | 5.2 | 5.3 | 5.4 | 5.5 | 5.6 |
|---|---|---|---|---|---|---|
| Glycerol | 4.98 | 4.98 | 4.98 | 4.98 | 4.98 | 4.98 |
| Caprylic/Capric triglyceride | 4.26 | 4.26 | 4.26 | 4.26 | 4.26 | 4.26 |
| Ethylhexyl palmitate | 4.00 | 4.00 | 4.00 | 4.00 | 4.00 | 4.00 |
| Carthamus tinctorius seed oil | 3.00 | 3.00 | 3.00 | 3.00 | 3.00 | 3.00 |
| Cocoglycerides | 3.00 | 3.00 | 3.00 | 3.00 | 3.00 | 3.00 |
| Sorbitol | 2.10 | 2.10 | 2.10 | 2.10 | 2.10 | 2.10 |
| Glyceryl oleate citrate | 1.24 | 1.24 | 1.24 | 1.24 | 1.24 | 1.24 |
| Aluminum starch | 0.93 | 0.93 | 0.93 | 0.93 | 0.93 | 0.93 |
| octenylsuccinate | | | | | | |
| Dimethicone | 0.50 | 0.50 | 0.50 | 0.50 | 0.50 | 0.50 |
| Carbomer | 0.30 | 0.30 | 0.30 | 0.30 | 0.30 | 0.30 |
| Fragrance | 0.28 | 0.28 | 0.28 | 0.28 | 0.28 | 0.28 |
| Sodium acrylate/sodium | 0.19 | 0.19 | 0.19 | 0.19 | 0.19 | 0.19 |
| acryloyldimethyl taurate | | | | | | |
| copolymer | | | | | | |
| Trilinolein | 0.16 | 0.16 | 0.16 | 0.16 | 0.16 | 0.16 |
| Polyisobutene | 0.14 | 0.14 | 0.14 | 0.14 | 0.14 | 0.14 |
| Ethyl lauroyl arginate * HCl | — | — | — | — | — | 0.40 |
| Piroctone olamine | 0.80 | 0.50 | — | — | — | 1.00 |
| Sodium sulfite | — | — | — | 0.20 | — | 0.20 |
| Hexetidine | 0.10 | 0.10 | 0.10 | — | 0.10 | 0.10 |
| Formic acid | — | — | — | — | — | 0.50 |
| Phenoxyisopropanol | — | — | — | 1.00 | — | 1.00 |
| Undecylenic acid | — | — | — | — | — | 0.20 |
| Chloroxylenol | — | — | — | — | 0.50 | 0.50 |
| Benzyl alcohol | — | — | — | — | — | 1.00 |
| Chlorphenesin | — | — | 0.30 | — | — | 0.30 |
| Benzoic acid | — | — | — | — | — | 0.20 |
| Phenoxyethanol | — | — | — | — | — | 0.50 |
| Methylparaben | — | 0.20 | — | — | — | 0.20 |
| Ethylparaben | — | — | — | — | — | 0.10 |
| Propionic acid | — | — | — | — | — | 2.00 |
| Sorbic acid | — | — | 0.60 | — | — | 0.60 |
| Polyaminopropyl biguanide | — | — | — | — | — | 0.30 |
| Climbazole | — | — | — | — | — | 0.50 |
| Chlorhexidine | — | — | — | — | — | 0.30 |
| Glutaraldehyde | — | — | — | — | — | 0.10 |
| Salicylic acid | 0.20 | — | 0.50 | — | — | 0.50 |
| Benzalkonium chloride | — | 0.10 | | — | — | 0.10 |
| KM¹⁾ | — | — | — | 0.3 | 1.2 | — |
| Panthenol | 0.08 | 0.08 | 0.08 | 0.08 | 0.08 | 0.08 |
| Tocopheryl acetate | 0.05 | 0.05 | 0.05 | 0.05 | 0.05 | 0.05 |
| Sorbitan oleate | 0.02 | 0.02 | 0.02 | 0.02 | 0.02 | 0.02 |
| Caprylyl/Capryl glucoside | 0.01 | 0.01 | 0.01 | 0.01 | 0.01 | 0.01 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Cleansing emulsion (all values represent wt %)

| Raw material | 6.1 | 6.2 | 6.3 | 6.4 | 6.5 | 6.6 |
|---|---|---|---|---|---|---|
| Glycerol | 5.01 | 5.01 | 5.01 | 5.01 | 5.01 | 5.01 |
| Sorbitol | 2.10 | 2.10 | 2.10 | 2.10 | 2.10 | 2.10 |
| Hexyl laurate | 2.00 | 2.00 | 2.00 | 2.00 | 2.00 | 2.00 |
| Ethylhexyl palmitate | 2.00 | 2.00 | 2.00 | 2.00 | 2.00 | 2.00 |
| Hydroxyethyl urea | 1.34 | 1.34 | 1.34 | 1.34 | 1.34 | 1.34 |
| Cetearyl alcohol | 1.00 | 1.00 | 1.00 | 1.00 | 1.00 | 1.00 |
| Aluminum starch | 0.93 | 0.93 | 0.93 | 0.93 | 0.93 | 0.93 |
| octenylsuccinate | | | | | | |
| Glyceryl stearate | 0.50 | 0.50 | 0.50 | 0.50 | 0.50 | 0.50 |
| Potassium cetyl phosphate | 0.31 | 0.31 | 0.31 | 0.31 | 0.31 | 0.31 |
| Carbomer | 0.30 | 0.30 | 0.30 | 0.30 | 0.30 | 0.30 |
| Fragrance | 0.19 | 0.19 | 0.19 | 0.19 | 0.19 | 0.19 |
| Hydrogenated palm glycerides | 0.19 | 0.19 | 0.19 | 0.19 | 0.19 | 0.19 |
| Sodium acrylate/sodium | 0.19 | 0.19 | 0.19 | 0.19 | 0.19 | 0.19 |
| acryloyldimethyl | | | | | | |
| taurate copolymer | | | | | | |
| Ethyl lauroyl arginate * HCl | — | — | 0.40 | 0.40 | — | 0.40 |
| Piroctone olamine | — | — | — | — | — | 1.00 |
| Sodium sulfite | — | — | — | — | 0.20 | 0.20 |
| Hexetidine | 0.10 | 0.10 | 0.10 | — | — | 0.10 |
| Formic acid | — | 0.50 | — | — | — | 0.50 |
| Phenoxyisopropanol | — | — | — | 1.00 | — | 1.00 |
| Undecylenic acid | — | — | — | — | — | 0.20 |
| Chloroxylenol | — | — | — | — | 0.50 | 0.50 |
| Benzyl alcohol | 0.50 | — | — | — | — | 1.00 |
| Chlorphenesin | — | — | — | — | — | 0.30 |
| Benzoic acid | — | 0.20 | — | — | — | 0.20 |
| Phenoxyethanol | — | — | — | — | — | 0.50 |
| Methylparaben | 0.10 | — | 0.20 | — | — | 0.20 |
| Ethylparaben | 0.15 | — | — | — | — | 0.10 |
| Propionic acid | | — | — | — | — | 2.00 |
| Sorbic acid | — | 0.20 | 0.20 | — | — | 0.60 |
| Polyaminopropyl biguanide | — | — | — | — | — | 0.30 |
| Climbazole | — | — | — | — | — | 0.50 |
| Chlorhexidine | — | 0.30 | — | — | — | 0.30 |
| Glutaraldehyde | — | — | — | — | — | 0.10 |
| Salicylic acid | — | — | — | — | — | 0.50 |
| Benzalkonium chloride | — | — | 0.10 | — | — | 0.10 |
| KM¹⁾ | — | — | — | 0.9 | 0.25 | — |
| Panthenol | 0.08 | 0.08 | 0.08 | 0.08 | 0.08 | 0.08 |
| Urea | 0.08 | 0.08 | 0.08 | 0.08 | 0.08 | 0.08 |
| Tocopheryl acetate | 0.05 | 0.05 | 0.05 | 0.05 | 0.05 | 0.05 |
| Sorbitan oleate | 0.02 | 0.02 | 0.02 | 0.02 | 0.02 | 0.02 |
| Caprylyl/Capryl glucoside | 0.01 | 0.01 | 0.01 | 0.01 | 0.01 | 0.01 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Cleansing milk (indicated in wt %)

| Raw material | 7.1 | 7.2 | 7.3 | 7.4 | 7.5 | 7.6 |
|---|---|---|---|---|---|---|
| Helianthus annuus seed oil | 5.00 | 5.00 | 5.00 | 5.00 | 5.00 | 5.00 |
| Isopropyl palmitate | 4.98 | 4.98 | 4.98 | 4.98 | 4.98 | 4.98 |
| Rosa damascena flower water | 4.97 | 4.97 | 4.97 | 4.97 | 4.97 | 4.97 |
| Ethyl lauroyl arginate * HCl | — | — | — | 0.40 | — | 0.40 |
| Piroctone olamine | — | — | — | — | — | 1.00 |
| Sodium sulfite | — | — | 0.20 | — | — | 0.20 |
| Hexetidine | — | — | — | — | 0.10 | 0.10 |
| Formic acid | 0.20 | — | — | 0.50 | — | 0.50 |
| Phenoxyisopropanol | — | — | — | — | — | 1.00 |
| Undecylenic acid | 0.20 | — | — | — | 0.20 | 0.20 |
| Chloroxylenol | — | 0.50 | — | — | — | 0.50 |
| Benzyl alcohol | — | 1.00 | — | — | — | 1.00 |
| Chlorphenesin | — | — | 0.30 | — | — | 0.30 |
| Benzoic acid | — | — | — | — | — | 0.20 |
| Phenoxyethanol | 0.30 | — | — | — | — | 0.50 |
| Methylparaben | — | — | — | — | — | 0.20 |
| Ethylparaben | — | — | — | — | — | 0.10 |
| Propionic acid | — | — | — | — | — | 2.00 |
| Sorbic acid | — | 0.60 | — | — | — | 0.60 |
| Polyaminopropyl biguanide | — | — | — | — | — | 0.30 |
| Climbazole | — | — | — | — | — | 0.50 |
| Chlorhexidine | — | 0.30 | | — | — | 0.30 |
| Glutaraldehyde | — | — | — | — | — | 0.10 |
| Salicylic acid | — | 0.20 | 0.20 | — | — | 0.50 |
| Benzalkonium chloride | — | 0.10 | — | — | — | 0.10 |
| KM¹⁾ | — | — | — | 0.7 | 0.01 | — |
| Cetearyl alcohol | 2.50 | 2.50 | 2.50 | 2.50 | 2.50 | 2.50 |
| Cocoglycerides | 2.00 | 2.00 | 2.00 | 2.00 | 2.00 | 2.00 |
| Laurus nobilis leaf extract | 1.99 | 1.99 | 1.99 | 1.99 | 1.99 | 1.99 |
| Hydrogenated vegetable oil | 1.00 | 1.00 | 1.00 | 1.00 | 1.00 | 1.00 |
| Xanthan gum | 1.00 | 1.00 | 1.00 | 1.00 | 1.00 | 1.00 |
| Lauryl glucoside | 0.95 | 0.95 | 0.95 | 0.95 | 0.95 | 0.95 |
| Polyglyceryl-2 | 0.95 | 0.95 | 0.95 | 0.95 | 0.95 | 0.95 |
| dipolyhydroxystearate | | | | | | |
| Panthenol | 0.50 | 0.50 | 0.50 | 0.50 | 0.50 | 0.50 |
| Citric acid | 0.21 | 0.21 | 0.21 | 0.21 | 0.21 | 0.21 |
| Sodium stearoyl glutamate | 0.10 | 0.10 | 0.10 | 0.10 | 0.10 | 0.10 |
| Tocopheryl acetate | 0.09 | 0.09 | 0.09 | 0.09 | 0.09 | 0.09 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Impregnation solution for cleaning cloths (amounts given in wt %)

| Raw material | 8.1 | 8.2 | 8.3 | 8.4 | 8.5 | 8.6 |
|---|---|---|---|---|---|---|
| Propylheptyl caprylate | 5.00 | 5.00 | 5.00 | 5.00 | 5.00 | 5.00 |
| Glycerol | 2.72 | 2.72 | 2.72 | 2.72 | 2.72 | 2.72 |
| Isopropyl palmitate | 2.00 | 2.00 | 2.00 | 2.00 | 2.00 | 2.00 |
| Lauryl glucoside | 1.36 | 1.36 | 1.36 | 1.36 | 1.36 | 1.36 |
| Polyglyceryl-2 | 1.36 | 1.36 | 1.36 | 1.36 | 1.36 | 1.36 |
| dipolyhydroxystearate | | | | | | |
| Caprylic/Capric triglyceride | 1.00 | 1.00 | 1.00 | 1.00 | 1.00 | 1.00 |
| Phenoxyethanol | 0.58 | 0.58 | 0.58 | 0.58 | 0.58 | 0.58 |
| Sodium polyacrylate | 0.35 | 0.35 | 0.35 | 0.35 | 0.35 | 0.35 |
| Fragrance | 0.29 | 0.29 | 0.29 | 0.29 | 0.29 | 0.29 |
| Ethyl lauroyl arginate * HCl | 0.40 | — | 0.40 | — | 0.4 | 0.40 |
| Piroctone olamine | — | — | — | — | — | 1.00 |
| Sodium sulfite | — | — | — | — | 0.20 | 0.20 |
| Hexetidine | 0.10 | 0.10 | — | — | — | 0.10 |
| Formic acid | — | — | — | 0.50 | — | 0.50 |
| Phenoxyisopropanol | — | — | — | — | — | 1.00 |
| Undecylenic acid | — | 0.20 | 0.20 | — | — | 0.20 |
| Chloroxylenol | — | — | — | — | — | 0.50 |
| Benzyl alcohol | — | — | — | 1.00 | — | 1.00 |
| Chlorphenesin | — | — | — | — | — | 0.30 |
| Benzoic acid | 0.20 | 0.20 | — | — | — | 0.20 |
| Phenoxyethanol | — | — | 0.50 | — | — | 0.50 |
| Methylparaben | — | — | — | — | — | 0.20 |
| Ethylparaben | — | — | — | — | — | 0.10 |
| Propionic acid | — | — | 2.00 | — | — | 2.00 |
| Sorbic acid | — | 0.60 | — | — | — | 0.60 |
| Polyaminopropyl biguanide | — | — | — | — | — | 0.30 |
| Climbazole | — | — | — | — | — | 0.50 |
| Chlorhexidine | — | 0.20 | 0.20 | — | — | 0.30 |
| Glutaraldehyde | — | — | — | — | — | 0.10 |
| Salicylic acid | — | — | — | — | — | 0.50 |
| Benzalkonium chloride | — | — | — | — | — | 0.10 |
| KM¹⁾ | — | — | — | 1.8 | 0.09 | — |
| Argania spinosa kernel oil | 0.10 | 0.10 | 0.10 | 0.10 | 0.10 | 0.10 |
| Citric acid | 0.03 | 0.03 | 0.03 | 0.03 | 0.03 | 0.03 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Impregnation solutions for cleaning cloths (amounts given in wt %)

| Raw material | 9.1 | 9.2 | 9.3 | 9.4 | 9.5 | 9.6 |
|---|---|---|---|---|---|---|
| Glycerol | 4.49 | 4.49 | 4.49 | 4.49 | 4.49 | 4.49 |
| Cetearyl isononanoate | 1.13 | 1.13 | 1.13 | 1.13 | 1.13 | 1.13 |
| Ceteareth-20 | 0.56 | 0.56 | 0.56 | 0.56 | 0.56 | 0.56 |
| Cetearyl alcohol | 0.56 | 0.56 | 0.56 | 0.56 | 0.56 | 0.56 |
| Potassium sorbate | 0.30 | 0.30 | 0.30 | 0.30 | 0.30 | 0.30 |
| Glyceryl stearate | 0.19 | 0.19 | 0.19 | 0.19 | 0.19 | 0.19 |
| Fragrance | 0.17 | 0.17 | 0.17 | 0.17 | 0.17 | 0.17 |
| Ethyl lauroyl | 0.40 | 0.40 | — | — | — | 0.40 |
| arginate * HCl | | | | | | |
| Piroctone olamine | — | — | — | — | 1.00 | 1.00 |
| Sodium sulfite | — | — | 0.20 | — | — | 0.20 |
| Hexetidine | — | — | — | — | — | 0.10 |
| Formic acid | — | — | 0.50 | — | — | 0.50 |
| Phenoxyisopropanol | — | — | — | 1.00 | — | 1.00 |
| Undecylenic acid | — | — | 0.20 | — | — | 0.20 |
| Chloroxylenol | 0.30 | — | — | — | — | 0.50 |
| Benzyl alcohol | — | — | — | 1.00 | — | 1.00 |
| Chlorphenesin | — | 0.30 | — | — | 0.30 | 0.30 |
| Benzoic acid | — | 0.20 | 0.20 | — | — | 0.20 |
| Phenoxyethanol | — | — | — | — | — | 0.50 |
| Methylparaben | — | — | — | — | — | 0.20 |
| Ethylparaben | — | 0.10 | — | — | — | 0.10 |
| Propionic acid | — | — | — | — | — | 2.00 |
| Sorbic acid | 0.20 | — | — | — | — | 0.60 |
| Polyaminopropyl | — | — | — | — | — | 0.30 |
| biguanide | | | | | | |
| Climbazole | — | — | 0.50 | — | — | 0.50 |
| Chlorhexidine | — | — | — | — | — | 0.30 |
| Glutaraldehyde | — | 0.10 | — | — | — | 0.10 |
| Salicylic acid | — | — | — | — | — | 0.50 |
| Benzalkonium | — | — | 0.10 | — | — | 0.10 |
| chloride | | | | | | |
| KM¹⁾ | — | — | — | 0.001 | 0.4 | — |
| Ceteareth-12 | 0.04 | 0.04 | 0.04 | 0.04 | 0.04 | 0.04 |
| Cetyl palmitate | 0.04 | 0.04 | 0.04 | 0.04 | 0.04 | 0.04 |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

Impregnation solution for cleaning cloths (amounts given in wt %)

| Raw material | 10.1 | 10.2 | 10.3 | 10.4 | 10.5 | 10.6 |
|---|---|---|---|---|---|---|
| Glycerol | 4.28 | 4.28 | 4.28 | 4.28 | 4.28 | 4.28 |
| Paraffinum liquidum | 4.00 | 4.00 | 4.00 | 4.00 | 4.00 | 4.00 |
| PPG-15 stearyl ether | 2.50 | 2.50 | 2.50 | 2.50 | 2.50 | 2.50 |
| Steareth-10 | 0.75 | 0.75 | 0.75 | 0.75 | 0.75 | 0.75 |
| Glyceryl stearate | 0.47 | 0.47 | 0.47 | 0.47 | 0.47 | 0.47 |
| PEG-30 STEARATE | 0.28 | 0.28 | 0.28 | 0.28 | 0.28 | 0.28 |
| Fragrance | 0.17 | 0.17 | 0.17 | 0.17 | 0.17 | 0.17 |
| Ethyl lauroyl | — | 0.40 | — | — | — | 0.40 |
| arginate * HCl | | | | | | |
| Piroctone olamine | 0.60 | — | — | — | 1.00 | 1.00 |
| Sodium sulfite | — | — | 0.20 | — | — | 0.20 |
| Hexetidine | 0.10 | — | — | — | — | 0.10 |
| Formic acid | — | — | 0.50 | — | — | 0.50 |
| Phenoxyisopropanol | — | — | — | 1.00 | — | 1.00 |
| Undecylenic acid | — | — | 0.20 | — | — | 0.20 |
| Chloroxylenol | — | — | — | — | — | 0.50 |
| Benzyl alcohol | — | — | — | 1.00 | — | 1.00 |
| Chlorphenesin | — | 0.30 | — | — | 0.30 | 0.30 |
| Benzoic acid | — | 0.20 | 0.20 | — | — | 0.20 |
| Phenoxyethanol | — | — | — | — | — | 0.50 |
| Methylparaben | — | — | — | — | — | 0.20 |
| Ethylparaben | — | 0.10 | — | — | — | 0.10 |
| Propionic acid | — | — | — | — | — | 2.00 |
| Sorbic acid | — | — | — | — | — | 0.60 |
| Dehydroacetic acid | 0.20 | 0.20 | 0.20 | — | — | 0.20 |
| Polyaminopropyl | 0.30 | — | — | — | — | 0.30 |
| biguanide | | | | | | |
| Climbazole | — | — | 0.50 | — | — | 0.50 |
| Chlorhexidine | — | — | — | — | — | 0.30 |
| Glutaraldehyde | — | 0.10 | — | — | — | 0.10 |
| Salicylic acid | — | — | — | — | — | 0.50 |
| Benzalkonium | — | — | 0.10 | — | — | 0.10 |
| chloride | | | | | | |
| KM¹⁾ | — | — | — | 0.001 | 0.4 | — |
| Aqua | up to | up to | up to | up to | up to | up to |
| | 100 | 100 | 100 | 100 | 100 | 100 |

¹⁾Preservative mixture selected from at least one of the aforementioned preservative mixtures KM1 to KM466

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent including, in a cosmetically acceptable carrier:
a) at least one emulsifier;
b) at least one compound selected from the group consisting of oils, waxes, esters, or mixtures thereof;
c) at least one preservative mixture selected from the group consisting of
chloroxylenol and phenoxyisopropanol,
undecylenic acid and formic acid,
phenoxyisopropanol and piroctone olamine,
phenoxyisopropanol and formic acid,
sulfite(s) and hexetidine,
ethyl lauroyl arginate and formic acid,
ethyl lauroyl arginate and chloroxylenol,
hexetidine and benzyl alcohol,
hexetidine and chloroxylenol,
hexetidine and piroctone olamine,
hexetidine and chlorophenesin,
hexetidine and formic acid,
and mixtures thereof, and
d) at least one further preservative selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof, and mixtures of these preservatives.

2. The cosmetic agent according to claim 1, wherein the at least one emulsifier is selected from the group consisting of: (i) addition products of 4 to 30 mol ethylene oxide and/or 1 to 5 mol propylene oxide with linear C₈-C₂₂ alcohols, with C₁₂-C₂₂ carboxylic acids, and with C₈-C₁₅ alkylphenols; (ii) C₁₂-C₂₂ carboxylic acid mono- and diesters of addition products of 1 to 30 mol ethylene oxide with C₃-C₆ polyols; (iii) ethylene oxide and polyglycerol addition products with methyl glucoside carboxylic acid esters, carboxylic acid alkanolamides, and carboxylic acid glucamides, C₈-C₂₂ alkylmono- and oligoglycosides; (iv) addition products of 5 to 60 mol ethylene oxide with castor oil and hydrogenated castor oil; (v) partial esters of polyols having three to six carbon atoms with saturated C₈-C₂₂ carboxylic acids; (vi) sterols; (vii) carboxylic acid esters of sugars and sugar alcohols; and (viii) mixtures thereof.

3. The cosmetic agent according to claim 1, including the oil, wherein the oil is selected from the group consisting of: (i) a volatile non-silicone oils selected from the group consisting of isodecane, isoundecane, isododecane, isotridecane, isotetradecane, isopentadecane, isohexadecane, and isoeicosane; (ii) vegetable oils selected from the group consisting of sunflower oil, olive oil, soybean oil, rapeseed oil, almond oil, jojoba oil, orange oil, wheat germ oil, peach kernel oil, and the liquid components of coconut oil; and (iii) mixtures thereof.

4. The cosmetic agent according to claim 1, including the wax, wherein the wax is selected from the group consisting of: (i); coconut fatty acid glycerol mono-, di-, and triesters; (ii) Butyrospermum parki (Shea butter); (iii) esters of saturated monohydric C₈-C₁₈ alcohols with saturated C₁₂-C₁₈ monocarboxylic acids; (iv) linear primary C₁₂-C₂₄ alkanols; (v) esters from a saturated monohydric C₁₆-C₆₀ alkanol and a saturated C₈-C₃₆ monocarboxylic acid; (vi) glycerol triesters of saturated linear C₁₂-C₃₀ carboxylic acids, which can be hydroxylated; (vii) natural vegetable waxes selected from the group consisting of candelilla wax, carnauba wax, Japan wax, sugar cane wax, ouricoury wax, cork wax, sunflower wax, and fruit waxes; (viii) animal waxes selected from the group consisting of bee wax, shellac wax, and spermaceti; (ix) synthetic waxes selected from the group consisting of montan ester waxes, hydrogenated jojoba waxes and sasol waxes, polyalkylene waxes, polyethylene glycol waxes, C₂₀-C₄₀ dialkyl esters of dimer acids, C₃₀₋₅₀ alkyl beeswax, and alkyl and alkyl aryl esters of dimeric fatty acids, paraffin waxes; and (x) mixtures thereof.

5. The cosmetic agent according to claim 1, including the ester, wherein the ester is selected from the group consisting of: (i) triethyl citrates; (ii) dicarboxylic acid esters of linear or branched C₂-C₁₀ alkanols; (iii) symmetric, asymmetric, or cyclic esters of carbonic acid with alcohols; (iv) esters of dimers of unsaturated C₁₂₋₂₂ carboxylic acids with monohydric, linear, branched, and cyclic C₂₋₁₈ alkanols or C₂₋₆ alkanols; (v) benzoic acid esters of linear or branched C₈₋₂₂ alkanols, such as benzoic acid C₁₂₋₁₅ alkyl esters, benzoic acid isostearyl esters, benzoic acid octyldodecyl esters; and (vi) mixtures thereof.

6. The cosmetic agent according to claim 1, wherein the emulsifier is included at a concentration of 0.1 to 40 wt % based on the total weight of the cosmetic agent.

7. The cosmetic agent according to claim 1, wherein the at least one compound selected from the group consisting of oils, waxes, esters, or mixtures thereof is included at a concentration of 0.1 to 50 wt % based on the total weight of the cosmetic agent.

8. The cosmetic agent according to claim 1, wherein a weight ratio of the first preservative to the second preservative in the preservative mixture c) ranges from 10:1 to 1:10.

9. The cosmetic agent according to claim 1, wherein the at least one preservative mixture c) is included at a concentration of 0.001 to 10 wt % based on the total weight of the cosmetic agent.

10. The cosmetic agent according to claim 1, further including at least one further preservative d) selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof.

11. The cosmetic agent according to claim 1, further including at least two further preservatives d) selected from the group consisting of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, citric acid and salts thereof.

12. The cosmetic agent according to claim 1, wherein a mixture of preservatives is included as the further preservative d), the mixture being made of benzoic acid and salts thereof, propionic acid and salts thereof, salicylic acid and salts thereof, sorbic acid and salts thereof, zinc salts, paraben(s), polyaminopropyl biguanide, phenoxyethanol, climbazole, chlorhexidine and salts thereof, quaternary ammonium compounds, glutaraldehyde, and citric acid.

13. The cosmetic agent according to claim 10, including the preservative mixture d) at a concentration of 0.001 to 10 wt % based on the total weight of the cosmetic agent.
